(19) United States
(12) Patent Application Publication
(10) Pub. No.: US 2024/0173235 A1
(43) Pub. Date: May 30, 2024

(54) PERSONAL CARE COMPOSITIONS COMPRISING SILAHYDROCARBONS

(71) Applicant: Momentive Performance Materials Inc., Waterford, NY (US)

(72) Inventors: Alok SARKAR, Bangalore (IN); Kenrick LEWIS, Flushing, NY (US); Benjamin FALK, Tarrytown, NY (US); Shrutisagar HAVELI, White Plains, NY (US); Manju Mamparambath DHARMAN, Bangalore (IN); Ashitha KANDIKKAL, Bangalore (IN)

(21) Appl. No.: 18/284,104

(22) PCT Filed: Apr. 13, 2022
(86) PCT No.: PCT/US2022/024522

§ 371 Date: Sep. 26, 2023

(30) Foreign Application Priority Data
Apr. 14, 2021 (IN) 202111017455

(51) Int. Cl. A61K 8/58; A61K 8/34; A61Q 1/02; A61Q 17/04

(52) U.S. Cl. CPC A61K 8/585; A61K 8/345; A61Q 1/02; A61Q 17/04; A61K 2800/31

(57) ABSTRACT

A personal care composition comprising (i) a solvent comprising at least one silahydrocarbon, and (ii) at least one personal care component. The inclusion of a silahydrocarbon material into a personal care composition can provide desirable volatility to such compositions and can impart enhanced sensory benefits and/or spreading characteristics to a personal care composition and personal care product.

### CROSS-REFERENCE TO RELATED APPLICATIONS

This application claims priority to and the benefit of India Patent Application 202111017455 filed on Apr. 14, 2021, the disclosure of which is incorporated herein by reference in its entirety.

### FIELD OF INVENTION

The present invention relates to a personal care composition. In particular, the present invention relates to a personal care composition comprising a solvent comprising a silahydrocarbon. The silahydrocarbon-based solvent provides personal care compositions with sensory associated properties as well as spreadability performance.

### BACKGROUND

Silicones are ingredients employed in most personal care formulations due to the light and smooth aesthetic benefits they offer. Certain cyclosiloxanes, namely octamethyltetracyclosiloxane (D4), and decamethylpentacyclosiloxane (D5), along with a few other low molecular weight linear siloxanes that offer excellent volatility profiles combined with their superior spreading and sensorial benefits, are among the most common silicones used in personal care formulations. These unique combinations of properties make these silicones highly attractive in the delivery of a variety of personal care products. There are generally a limited number of silicones available that can meet the volatility criteria requirement for personal care applications. Further, certain regulatory agencies have identified cyclosiloxanes such as D4 and D5 as substances of concern as bioaccumulating, persistent, and/or toxic. Thus, the use of such compounds in personal care compositions may be limited in the future such that other substances will be required to provide the desired sensory and spreading properties in personal care compositions.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Provided is a personal care composition comprising at least one silahydrocarbon and at least one personal care component. The incorporation of a silahydrocarbon into a personal care composition has been found to provide a personal care composition with useful properties including, but not limited to, suitable volatility and spreading properties.

In one aspect, provided is a personal care composition comprising (i) a solvent comprising at least one silahydrocarbon, and (ii) at least one personal care component.

In one embodiment, the silahydrocarbon is selected from a compound of the formula (I), formula (II), or a mixture of such silahydrocarbons:

R¹R²R³R⁴Si (I); or

R⁵R⁶R⁷Si(Q)SiR⁸R⁹R¹⁰ (II)

wherein R¹, R², R³, R⁴, R⁵, R⁶, R⁷, R⁸, R⁹, and R¹⁰ are each independently selected from an aliphatic hydrocarbyl group having 1 to 30 carbon atoms, an aryl group having 6 to 30 carbon atoms, an alkaryl having 7 to 30 carbon atoms, and a cycloaliphatic hydrocarbyl group having from 4 to 30 carbon atoms, and Q is a group bridging between silicon atoms.

In one embodiment, Q is selected from a straight-chained or branched alkylene group having from 0 to 14 carbon atoms, a cyclic hydrocarbon having 4 to 14 carbon atoms, and an aryl group having 6 to 14 carbon atoms.

In one embodiment of the personal care composition of any previous embodiment, the silahydrocarbon has a total number of carbons of about 30 or less.

In one embodiment of the personal care composition of any previous embodiment, the silahydrocarbon has a total number of carbons of about 20 or less.

In one embodiment of the personal care composition of any previous embodiment, the silahydrocarbon has a total number of carbon atoms of from about 5 to about 30.

In one embodiment of the personal care composition of any previous embodiment, the silahydrocarbon has a total number of carbon atoms of from about 7 to about 20.

In one embodiment of the personal care composition of any previous embodiment, the silahydrocarbon is selected from Trimethyl(butyl)silane; Trimethyl(pentyl)silane; Trimethyl(hexyl)silane; Trimethyl(heptyl)silane; Trimethyl(octyl)silane; Trimethyl(nonyl) silane; Trimethyl(decyl)silane; Trimethyl(undecyl)silane; Trimethyl(dodecyl)silane; Ethyldimethyl(butyl)silane; Ethyldimethyl(pentyl)silane; Ethyldimethyl(hexyl)silane; Ethyldimethyl(heptyl)silane; Ethyldimethyl (octyl)silane; Ethyldimethyl (nonyl) silane; Ethyldimethyl (decyl)silane; Ethyldimethyl (undecyl)silane; Ethyldimethyl (dodecyl)silane; Diethylmethyl(butyl)silane; Diethylmethyl (pentyl)silane; Diethylmethyl (hexyl)silane; Diethylmethyl (heptyl)silane; Diethylmethyl (octyl)silane; Diethylmethyl (nonyl) silane; Diethylmethyl (decyl)silane; Diethylmethyl (undecyl)silane; Diethylmethyl (dodecyl)silane; Triethyl (butyl); Triethyl (pentyl)silane; Triethyl (hexyl)silane; Triethyl (heptyl)silane; Triethyl (octyl)silane; Triethyl (nonyl) silane; Triethyl (decyl)silane; Triethyl (undecyl)silane; Triethyl (dodecyl)silane; Tetraethylsilane; Tetrabutylsilane [R¹1=R²=R³=R⁴=butyl](16 carbon atoms); Tributylmethylsilane; Tributylethylsilane; Dibutyldimethylsilane; Dibutyldiethylsilane; Tetrapropylsilane; Tripropylmethylsilane; Tripropylethylsilane; Dipropyldimethylsilane; Dipropyldiethylsilane; 2,4,4-Trimethylpentyltriethylsilane; Hexamethyldisilane, Bis(trimethylsilyl)methane, Bis(trimethylsilyl)ethane; Bis(trimethylsilyl)butane; Bis(trimethylsilyl)pentane; Bis(trimethylsilyl)hexane; Bis(trimethylsilyl)heptane; Bis(trimethylsilyl) octane; Bis(triethylsilyl)butane; Bis(triethylsilyl)pentane; Bis(triethylsilyl)hexane; Bis(triethylsilyl)heptane; Bis(triethylsilyl)octane; 1-Dimethylbutylsilyl 8-trimethylsilyl octane; or a combination of two or more thereof.

In one embodiment of the personal care composition of any previous embodiment, the silahydrocarbon is present in the formulation in an amount of from about 40% to about 100% by weight based on the total weight of the composition.

In one embodiment of the personal care composition of any previous embodiment, the silahydrocarbon is present in an amount of from about 1% to about 80% by weight based on the total weight of the personal care composition.

In one embodiment of the personal care composition of any previous embodiment, the silahydrocarbon is present in an amount of from about 5% to about 60% by weight based on the total weight of the personal care composition.

In one embodiment of the personal care composition of any previous embodiment, the silahydrocarbon is present in an amount of from about 10% to about 50% by weight based on the total weight of the personal care composition.

In one embodiment of the personal care composition of any previous embodiment, at least one personal care component is selected from a gel forming polymer, a film forming polymer, an emulsion, an elastomer, a resin, a wax, an oil, an alcohol, an ester, a pigment, a bio-active agent, a sunscreen agent, or a combination of two or more thereof.

T In one embodiment of the personal care composition of any previous embodiment, the gel forming polymer is selected from a crosslinked silicone gel, a crosslinked acrylamide gel, a crosslinked hydrocarbon gel, a functionalized cellulose gel, or a combination of two or more thereof.

In one embodiment of the personal care composition of any previous embodiment, the wax is selected from beeswax, paraffin, rice bran wax, candelilla wax, carnauba wax, ozokerite wax, or a combination of two or more thereof.

In one embodiment of the personal care composition of any previous embodiment, the emulsion is selected from a lamellar emulsion, a microemulsion, a nanoemulsion, a fluid simple emulsion, a fluid multiple emulsion, a rigid simple emulsion, or a rigid multiple emulsion.

In one embodiment of the personal care composition of any previous embodiment, the emulsion is an oil-in-water emulsion or a water-in-oil emulsion.

In one embodiment of the personal care composition of any previous embodiment, the hydrocarbon is selected from polybutene, polyisobutene, polycyclopentadiene, isododecane, isohexadecane, petroleum jelly, mineral oil, or a combination of two or more thereof.

In one embodiment of the personal care composition of any previous embodiment, the oil is selected from jojoba oil, Macadamia nut oil, almond oil, avocado oil, sunflower oil, thistle oil, castor oil, apricot kernel oil, peach kernel oil, coconut oil, palm oil, olive oil, sesame oil, soybean oil, and one or more active substance oils, preferably selected from the group argon oil, Rosehip Oil (Rose hip seed oil), Evening primrose oil, Borage seed oil, Camellias oil, Amaranth seed oil, Babussaöl, Rice bran oil, Grapeseed oil, walnut oil, wheat germ oil, cottonseed oil, calendula oil, hemp oil, currant seed oil, kukui nut oil, laurel oil, poppy seed oil, black cumin oil, or a combination of two or more thereof.

In one embodiment of the personal care composition of any previous embodiment, the alcohol is selected from glycerin, glycol, butylene glycol, propylene glycol, pentylene glycol, cetyl alcohol, propyelene glycol ethanol, polyvinyl alcohol, phenoxyethanol, isononyl alcohol, neopentyl alcohol, or a combination of two or more thereof

In one embodiment of the personal care composition of any previous embodiment, the personal care component is selected from a dimethicone polymer, a dimethicone copolymer, a polyvinyl alcohol copolymer, a polyacrylate copolymer, a polyvinyl pyrrolidone copolymer, a crosslinked vinyl dimethicone polymer, a silicone elastomer, a polymethylmethacrylate elastomer, ispropyl myristate, isopropyl palmitate, or a combination of two or more thereof.

In one embodiment, the personal care component is selected from a vitamin, skin nourishing agent, hair nourishing agent, antidandruff additive, antibacterial, antifungal agent, or combination of two or more thereof.

In one embodiment, the a vitamin, skin nourishing agent, hair nourishing agent, antidandruff additive, antibacterial, or antifungal agent is selected form ceramides, hyaluronic acid, panthenol, peptides (copper hexapeptide-3), AHAs (lactic acid), retinols (retinyl palmitate)-vitamin A derivatives, vitamin C (1-ascorbic acid), BHAs (salicylic acid), niacinamide, tocopherol, teas (green tea, white tea, red tea), soy and other plant derivatives, isoflavones (grape seed extract), argireline, acai berry, pyridinethione salts, 1-hydroxy-2-pyrrolidone derivatives, 2,2′-dithiobis(pyridine-N-oxide), trihalocarbamides, triclosan, an azole compound, selenium sulphides, extracts of one or more non-photosynthetic, non-fruiting filamentous bacteria, zinc pyrithione, piroctone olamine, selenium disulphide, sulphur, coal tar, or a combination of two or more thereof.

In one embodiment of the personal care composition of any previous embodiment, the sunscreen agent is selected from p-aminobenzoic acid, avobenzone cinoxate, dioxybenzone, homosalate, menthyl anthranilate, octocrylene, octyl methoxycinnamate, octyl salicylate, oxybenzone, padimate, phenylbenzimidazole sulfonic acids, sulisobenzone, trolamine salicylate, aminobenzoic acid, amyldimethyl p-aminobenzoic acid, diethanolamine p-methoxycinnamate, digalloyl trioleate, 2-ethylhexyl-2-cyano-3,3-diphenylacrylate, ethylhexyl p-methoxycinnamate, 2-ethylhexyl salicylate, glyceryl aminobenzoate, homomethyl salicylate, homosalate, 3-imidazol-4-ylacrylic acid and the ethyl ester thereof, methyl anthranilate, octyldimethyl PABA, 2-phenylbenzimidazole-5-sulfonic acid and salts, sulisobenzone, triethanolamine salicylate, N,N,N-trimethyl-4-(2-oxoborn-3-ylidene methyl)anillinium methyl sulfate, aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammonium)-benzyliden-boman-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′ dimethoxybenzophenone, 2-hydroxy-4-n-octoxybenzophenone, 2-hydroxy-4-methoxy-4′-methoxybenzophenone, ca-(2-oxoborn-3-ylidene)-tolyl-4-sulfonic acid and soluble salts thereof, 3-(4′-sulfo)benzyliden-boman-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris-(2′-ethylhexyl-1′-oxycarbonyl)-anilinol 1,3,5-triazine, 2-(p-(tert-butylamido)anilinol-4,6-bis-(p-(2′-ethylhexyl 1′-oxycarbonyl)anilinol 1,3,5-triazine, 2,4-bis{1,4-(2-ethylhexyloxy)-2-hydroxyl-phenyl}-6-(4-methoxyphenyl)-1,3,5-triazine, the polymer of N-(2 et 4)-(2-oxoborn-3-yliden)methylbenzyl acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (drometrizole trisiloxane), solubilized 2,2′-methylene-bis-1,6-(2H-benzotriazol-2-yl)-4-(1,1,3,3-tetramethylbutyl)phenol, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, or a combination of two or more thereof.

In one embodiment of the personal care composition of any previous embodiment, the pigment is selected from a pearl, titanium oxide, Red 6, Red 21, Blue 1, Orange 5, Green 5, chalk, talc, iron oxide, boron nitride, zinc oxide, magnesium oxide, titanated mica, or a combination of two or more thereof.

In one embodiment of the personal care composition of any previous embodiment, the personal care composition is selected from deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail- and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, drug delivery systems for topical application of medicinal compositions that are to be applied to the skin.

In another aspect, provided is a method of treating a biological substrate comprising applying the personal care composition of any of claims 1-25 to a surface of the biological substrate.

In one embodiment, the biological substrate is skin or hair.

The following description and the drawings disclose various illustrative aspects. Some improvements and novel aspects may be expressly identified, while others may be apparent from the description and drawings.

### BRIEF DESCRIPTION OF THE DRAWINGS

FIG. 1 is a graph comparing the volatility of silahydrocarbons in accordance with the invention with that of cyclopentasiloxane; and

FIG. 2 is a chart comparing the spreadability of a silahydrocarbon with cyclopentasiloxane.

### DETAILED DESCRIPTION

Reference will now be made to exemplary embodiments, examples of which are illustrated in the accompanying drawings. It is to be understood that other embodiments may be utilized, and structural and functional changes may be made. Moreover, features of the various embodiments may be combined or altered. As such, the following description is presented by way of illustration only and should not limit in any way the various alternatives and modifications that may be made to the illustrated embodiments. In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

The disclosure may identify a number of different ranges for a component or components in the composition. It will be appreciated that the numerical values of the respective ranges can be combined to form new and non-specified ranges.

The term a “personal care composition,” as used herein, is a composition comprising at least one silahydrocarbon compound as described herein and at least one personal care component or ingredient of a personal care application.

The term a “personal care component” includes a functional agent or material suitable to provide a desired type of personal care product or application.

A “personal care application” as used herein is understood to be a consumer product used for personal hygiene and/or personal beautification. A personal care application can be used interchangeably with a personal care component.

The term “alkyl” includes straight, branched, and cyclic monovalent hydrocarbon groups, which may be substituted with a heteroatom or heteroatom containing group. In embodiments, the term alkyl may include C1-C30 alkyl groups. Examples of suitable alkyl groups include, but are not limited to, methyl, ethyl, propyl, isopropyl, butyl, isobutyl, tertbutyl, pentyl, hexyl, heptyl, octyl, nonyl, decyl, etc.

The term “alkylene” includes straight, branched, and cyclic divalent hydrocarbon groups, which may be substituted with a heteroatom or heteroatom containing group. In embodiments, the term alkylene includes C1-C30 alkylene groups. Examples of alkylenes include, but are not limited to, methylene, ethylene, propylene, isopropylene, butylene, isobutylene, tertbutylene, pentylene, hexylene, heptylene, octylene, nonylene, decylene, etc.

The term “aryl” includes any monovalent aromatic hydrocarbon group, which may be substituted with a heteroatom or heteroatom containing group. This term also includes fused systems containing an aromatic group and groups with multiple aryl groups joined by a bond or linker group. In embodiments, the term aryl include C6-C30 aryl groups, fused aryl groups comprising two or more C6-C30 aryl groups, and multi-aryl group structures comprising two or more C6-C30 aryl groups joined by a linker group. Examples of aryl groups include, but are not limited to, phenyl, tolyl, xylyl, trimethylphenyl, phenylethyl, phenylpropyl, phenylbutyl, etc.

The term “arylene” includes any divalent aromatic hydrocarbon group, which may be substituted with a heteroatom or heteroatom containing group this term also includes fused systems containing an aromatic group. In embodiments, the term aryl include C6-C30 arylene groups, fused arylene groups comprising two or more C6-C30 aryl groups, and multi-arylene group structures comprising two or more C6-C30 aryl groups joined by a linker group.

The term “aralkyl” include straight, branched, and cyclic monovalent hydrocarbon groups substituted with an aryl substituent.

The term “cyclo” or “cyclic” alkyl includes a monovalent cyclic hydrocarbon and includes, free cyclic groups, bicyclic groups, tricyclic groups, and higher cyclic structures, as well as bridged cyclic groups, fused cyclic groups, and fused cyclic groups containing at least one bridged cyclic group. In embodiments, a cyclic alkyl includes a C3-C20 cyclic alkyl group. Example of suitable cyclic groups include, but are not limited to, cyclopropyl, cyclopentyl, cyclohexyl, norbornyl, bicyclo[2.2.2]nonane, adamantyl, or tetrahydronaphthyl (tetralin).

The term “cyclo” or “cyclic” alkylene includes a divalent cyclic hydrocarbon and includes, free cyclic groups, bicyclic groups, tricyclic groups, and higher cyclic structures, as well as bridged cyclic groups, fused cyclic groups, and fused cyclic groups containing at least one bridged cyclic group. In embodiments, a cyclic alkylene includes a C3-C20 cyclic alkylene group.

The term “alkynyl” is defined as a C2-C10 branched or straight-chain unsaturated aliphatic hydrocarbon groups having one or more triple bonds between two or more carbon atoms. Examples of alkynes include ethynyl, propynyl, butynyl, pentynyl, hexynyl, heptynyl, octynyl and nonynyl.

The term “substituted” means that one or more hydrogens on the molecule, portion of the molecule, or atom are replaced by a substitution group provided that the normal valency is not exceeded. The substitution group can be a heteroatom. The term “hetero” as used refer to an atom or in conjunction with another group includes an atom or group containing an atom such as oxygen, nitrogen, sulfur, silicon, phosphorus, boron, etc. Examples of suitable substitution groups include, but are not limited to, —OR, —NR′R, —C(O)R, —SR, -halo, —CN, —NO₂,—SO₂, phosphoryl, imino, thioester, carbocyclic, aryl, heteroaryl, alkyl, alkenyl, bicyclic and tricyclic groups. When a substitution group is a keto (i.e., ═O) group, then 2 hydrogens on the atom are replaced. Keto substituents are not present on aromatic moieties. The terms R and R′ refer to alkyl groups that may be the same or different.

Provided is a personal care composition comprising (i) at least one solvent comprising a silahydrocarbon, and (ii) at least one personal care component. The silahydrocarbons have been found to be suitable replacements for conventional solvents such as, for example, D4 (octamethyltetracyclosilxoane) or D5 (decamethylpentacyclosiloxane). The use of silahydrocarbons in a personal care composition has been found to provide compositions with desirable properties including desired sensory properties as well as spreading properties (such as spreading when applied to the surface of the skin).

The silahydrocarbon employed in the personal care compositions is chosen from a silahydrocarbon of the formula (I), formula (II), or a mixture of such silahydrocarbons:

R¹R²R³R⁴Si (I); or

R⁵R⁶R⁷Si(Q)SiR⁸R⁹R¹⁰ (II)

wherein R¹, R², R³, R⁴, R⁵, R⁶, R⁷, R⁸, R⁹, and R¹⁰ are each independently selected from an aliphatic hydrocarbyl group having 1 to 30 carbon atoms, an aryl group having 6 to 30 carbon atoms, an alkaryl having 7 to 30 carbon atoms, and a cycloaliphatic hydrocarbyl group having from 4 to 30 carbon atoms, and Q is a group bridging between silicon atoms. In embodiments, Q is selected from a straight-chained or branched alkylene group having from 0 to 14 carbon atoms, a cyclic hydrocarbon having 4 to 14 carbon atoms, and an aryl group having 6 to 14 carbon atoms. Compounds of the formula (II) may be referred to herein as polysilahydrocarbon compounds.

In one embodiment, R¹, R², R³, R⁴, R⁵, R⁶, R⁷, R⁸, R⁹, and R¹⁰ are each independently selected from an aliphatic hydrocarbyl group having 1 to 20 carbon atoms, an aryl group having 6 to 20 carbon atoms, an alkaryl having 7 to 20 carbon atoms, and a cycloaliphatic hydrocarbyl group having from 4 to 20 carbon atoms; In another embodiment, R¹, R², R³, R⁴, R⁵, R⁶, R⁷, R⁸, R⁹, and R¹are each independently selected from an aliphatic hydrocarbyl group having 2 to 16 carbon atoms, an aryl group having 8 to 16 carbon atoms, an alkaryl having 7 to 14 carbon atoms, and a cycloaliphatic hydrocarbyl group having from 6 to 16 carbon atoms.

In one embodiment, R¹, R², R³, R⁴, R⁵, R⁶, R⁷, R⁸, R⁹, and R¹⁰ are each independently selected from an aliphatic hydrocarbon having 1 to 12 carbon atoms, 2 to 10 carbon atoms 4 to 8 carbon atoms, or 5 to 6 carbon atoms.

Examples of suitable groups for R¹, R², R³, R⁴, R⁵, R⁶, R⁷, R⁸, R⁹, and R¹⁰include, but are not limited to, methyl, ethyl, propyl, isopropyl, butyl, isobutyl, t-butyl, pentyl, isopentyl, hexyl, heptyl, octyl, nonyl, decyl, octadecyl, phenyl, phenylethyl, cyclopentyl, cyclohexyl, cyclohexylpropyl, etc.

The total number of carbon atoms in the silahydrocarbons employed in the present compositions should at least be sufficient to provide liquid compounds at ambient temperature and would volatilize at temperatures below 100° C. In embodiments, the silahydrocarbons of formula (I) and (II) each have a total number of carbons of 30 or less; 25 or less; 20 or less; 15 or less; or 10 or less. In embodiments, the total number of carbon atoms in the silahydrocarbon material is 5 to 30, 7 to 20, 8 to 18, or 10 to 15. It will be appreciated that the minimum number of carbon atoms in a compound of formula (II) will be 6.

Examples of suitable silahydrocarbons of the formula (I) include, but are not limited to, Trimethyl(butyl)silane [R¹=R²=R³=methyl, R⁴=butyl](7 carbon atoms); Trimethyl(pentyl)silane [R¹=R²=R³=methyl, R⁴=pentyl](8 carbon atoms); Trimethyl(hexyl)silane [R¹=R²=R³=methyl, R⁴=hexyl](9 carbon atoms); Trimethyl(heptyl)silane [R¹=R²=R³=methyl, R⁴=heptyl](10 carbon atoms); Trimethyl(octyl)silane [R¹=R²=R³=methyl, R⁴=octyl](11 carbon atoms); Trimethyl(nonyl) silane [R¹=R²=R³=methyl, R⁴=nonyl](12 carbon atoms); Trimethyl(decyl)silane [R¹=R²=R³=methyl, R⁴=decyl](13 carbon atoms); Trimethyl(undecyl)silane [R¹=R²=R³=methyl, R⁴=undecyl](14 carbon atoms); Trimethyl(dodecyl)silane [R¹=R²=R³=methyl, R⁴=dodecyl](15 carbon atoms); Ethyldimethyl(butyl)silane [R¹=Ethyl, R²=R³=methyl, R⁴=butyl](8 carbon atoms); Ethyldimethyl(pentyl)silane [R¹=Ethyl, R²=R³=methyl, R⁴=pentyl](9 carbon atoms); Ethyldimethyl (hexyl)silane [R¹=Ethyl, R²=R³=methyl, R⁴=hexyl](10 carbon atoms); Ethyldimethyl (heptyl)silane [R¹=Ethyl, R²=R³=methyl, R⁴=heptyl](11 carbon atoms); Ethyldimethyl (octyl)silane [R¹=Ethyl, R²=R³=methyl, R⁴=octyl](12 carbon atoms); Ethyldimethyl (nonyl) silane [R¹=Ethyl, R²=R³=methyl, R⁴=nonyl](13 carbon atoms); Ethyldimethyl (decyl)silane [R¹=Ethyl, R²=R³=methyl, R⁴=decyl](14 carbon atoms); Ethyldimethyl (undecyl)silane [R¹=Ethyl, R²=R³=methyl, R⁴=undecyl](15 carbon atoms); Ethyldimethyl (dodecyl)silane [R¹=Ethyl, R²=R³=methyl, R⁴=dodecyl](16 carbon atoms); Diethylmethyl(butyl)silane [R¹=R²=Ethyl, R³=methyl, R⁴=butyl](9 carbon atoms); Diethylmethyl (pentyl)silane [R¹=R²=Ethyl, R³=methyl, R⁴=pentyl](10 carbon atoms); Diethylmethyl (hexyl)silane [R¹=R²=Ethyl, R³=methyl, R⁴=hexyl](11 carbon atoms); Diethylmethyl (heptyl)silane [R¹=R²=Ethyl, R³=methyl, R⁴=heptyl](12 carbon atoms); Diethylmethyl (octyl)silane [R¹=R²=Ethyl, R³=methyl, R⁴=octyl](13 carbon atoms); Diethylmethyl (nonyl) silane [R¹=R²=Ethyl, R³=methyl, R⁴=nonyl](14 carbon atoms); Diethylmethyl (decyl)silane [R¹=R²=Ethyl, R³=methyl, R⁴=decyl](15 carbon atoms); Diethylmethyl (undecyl)silane [R¹=R²=Ethyl, R³=methyl, R⁴=undecyl](16 carbon atoms); Diethylmethyl (dodecyl)silane [R¹=R²=Ethyl, R³=methyl, R⁴=dodecyl](17 carbon atoms); Triethyl (butyl)silane [R¹=R²=R³=ethyl, R⁴=butyl](10 carbon atoms); Triethyl (pentyl)silane [R¹=R²=R³=ethyl, R⁴=pentyl](11 carbon atoms); Triethyl (hexyl)silane [R¹=R²=R³=ethyl, R⁴=hexyl](12 carbon atoms); Triethyl (heptyl)silane [R¹=R²=R³=ethyl, R⁴=heptyl](13 carbon atoms); Triethyl (octyl)silane [R¹=R²=R³=ethyl, R⁴=octyl](14 carbon atoms); Triethyl (nonyl) silane [R¹=R²=R³=ethyl, R⁴=nonyl](15 carbon atoms); Triethyl (decyl)silane [R¹=R²=R³=ethyl, R⁴=decyl](16 carbon atoms); Triethyl (undecyl)silane [R¹=R²=R³=ethyl, R⁴=undecyl](17 carbon atoms); Triethyl (dodecyl)silane [R¹=R²=R³=ethyl, R⁴=dodecyl](18 carbon atoms); Tetraethylsilane [R¹=R²=R³=R⁴=ethyl](8 carbon atoms); Tetrabutylsilane [R¹=R²=R³=R⁴=butyl](16 carbon atoms); Tributylmethylsilane [R¹=R²=R³=butyl, R⁴=methyl](13 carbon atoms); Tributylethylsilane [R¹=R²=R³=butyl, R⁴=ethyl](14 carbon atoms); Dibutyldimethylsilane [R¹=R²=butyl, R³=R⁴=methyl](10 carbon atoms); Dibutyldiethylsilane [R¹=R²=butyl, R³=R⁴=ethyl]((12 carbon atoms); Tetrapropylsilane [R¹=R²=R³=R⁴=propyl](12 carbon atoms); Tripropylmethylsilane [R¹=R²=R³=propyl, R⁴=methyl](10 carbon atoms); Tripropylethylsilane [R¹=R²=R³=propyl, R⁴=ethyl](11 carbon atoms); Dipropyldimethylsilane [R¹=R²=propyl, R³=R⁴=methyl](8 carbon atoms); Dipropyldiethylsilane [R¹=R²=propyl, R³=R⁴=ethyl](10 carbon atoms); 2,4,4-Trimethylpentyltriethylsilane[R¹=R²=R³=Ethyl, R⁴=2,4,4-Trimethylpentyl](14 carbon atoms)

Examples of suitable polysilahydrocarbons of the formula (II) include, but are not limited to, Hexamethyldisilane [R(⁵=R⁶=R⁷=R⁸=R⁹=R¹⁰=methyl, Q=—(CH₂)₀—](6 carbon atoms); Bis(trimethylsilyl)methane [R⁵=R⁶=R⁷=R⁸=R⁹=R¹⁰=methyl, Q=—(CH₂)₁—](7 carbon atoms); Bis(trimethylsilyl)ethane [R⁵=R⁶=R⁷=R⁸=R⁹=R¹⁰=methyl, Q=—(CH₂)₂—](8 carbon atoms); Bis(trimethylsilyl)butane [R⁵=R⁶=R⁷=R⁸=R⁹=R¹⁰=methyl, Q=—(CH₂)₄—](10 carbon atoms); Bis(trimethylsilyl)pentane [R⁵=R⁶=R⁷=R⁸=R⁹=R¹⁰=methyl, Q=—(CH₂)₅—](11 carbon atoms); Bis(trimethylsilyl)hexane [R⁵=R⁶=R⁷=R⁸=R⁹=R¹⁰=methyl, Q=—(CH₂)₆—](12 carbon atoms); Bis(trimethylsilyl)heptane [R⁵=R⁶=R⁷=R⁸=R⁹=R¹⁰=methyl, Q=—(CH₂)₇—](13 carbon atoms); Bis(trimethylsilyl) octane [R⁵=R⁶=R⁷=R⁸=R⁹=R¹⁰=methyl, Q=—(CH₂)₈—](14 carbon atoms); Bis(triethylsilyl)butane [R⁵=R⁶=R⁷=R⁸=R⁹=R¹⁰=ethyl, Q=—(CH₂)₄—](16 carbon atoms); Bis(triethylsilyl)pentane [R⁵=R⁶=R⁷=R⁸=R⁹=R¹⁰=ethyl, Q=—(CH₂)₅—](17 carbon atoms); Bis(triethylsilyl)hexane [R⁵=R⁶=R⁷=R⁸=R⁹=R¹⁰=ethyl, Q=—(CH₂)₆—](18 carbon atoms); Bis(triethylsilyl)heptane [R⁵=R⁶=R⁷=R⁸=R⁹=R¹⁰=ethyl, Q=—(CH₂)₇—](19 carbon atoms); Bis(triethylsilyl)octane [R⁵=R⁶=R⁷=R⁸=R⁹=R¹⁰=ethyl, Q=—(CH₂)₈—](20 carbon atoms); and 1-Dimethylbutylsilyl 8-trimethylsilyl octane [R⁵=R⁶=R⁷=R⁸=R⁹=methyl, R¹⁰=butyl, Q=—(CH₂)₈—](17 carbon atoms)

Silahydrocarbons and polysilahydrocarbons can be synthesized via any suitable methods and reagents now known, or later discovered, to make silahydrocarbon materials. Examples of suitable methods and reagents to produce silahydrocarbons include, but are not limited to, hydrosilylation, Grignard reactions, redistribution, alkyl lithium reagents, trialkylsilyl potassium, trialkylsilyl sodium reagents, and the like. In addition, some synthetic methods employ Green Chemistry Principles (Anastas, P. T.; Warner, J. C. Green Chemistry: Theory and Practice, Oxford University Press: New York, 1998, p.30.) and also utilize raw materials derived from plants to highlight the sustainability benefits obtainable with silahydrocarbons. The silahydrocarbons of formula (II) with Q═0 can be synthesized according to a method described in reference: David E Seitz & Lawrence Ferreira (1979) An Efficient Preparation of Hexamethyldisilane, Synthetic Communications, 9:5, 451-456.

Hydrosilylation reactions are addition reactions. They have one hundred percent atom economy, high effective mass yields, and low E Factors. Hydrosilylation reactions can be done with or without solvents and with a variety of catalysts. Conventional hydrosilylation catalysts include Pt-based catalysts such as Karstedt's, Speier, or Lamaroeux catalysts, as well as with free radical generators and metal carbonyls assisted with UV-VIS irradiation. The olefins can be obtained from petroleum sources, but are preferably obtained by means fermentation and other biocatalytic processes when the silahydrocarbons are used in personal care. Octyltrimethylsilane, Dibutyldimethylsilane and Heptyltriethylsilane are non-limiting examples of silahydrocarbons that can be synthesized via Pt-catalyzed or free radical hydrosilylation. The equations show the anti-Markovnikov isomers, but one skilled in the art will recognize that formation of the Markovnikov isomers is also possible.

(CH₃)₃SiH+C₇H₁₃CH═CH₂ →(CH₃)₃SiC₈H₁₇ (Octyltrimethylsilane)

(CH₃)₂SiH₂ +2 C₃H₅CH═CH₂ →(CH₃)₂Si(C₄H₉)₂ (Di n-butyldimethylsilane)

(C₂H₅)₃SiH+C₆H₁₁CH═CH₂ →(C₂H₅)₃SiC₇H₁₅ (Heptyltriethylsilane)

(CH₃)₃SiCH═CH₂+(CH₃)₃SiC₂H₄Si(CH₃)₂H→(CH₃)₃SiC₂H₄Si(CH₃)₂CH₂CH₂Si(CH₃)₃

In U.S. Pat. No. 9,371,399, silahydrocarbons are synthesized with the use of iron pyridine diimine complexes such as the μ-dinitrogen dimer, [(^{Me}PDI)FeN₂]₂(μ₂—N₂), μ denotes bridging N₂ ligand, [(^{Me}PDI)=2,6(2,6-Me₂-C₆H₃N=CMe)₂C₅H₃N, Me=CH₃. Dimethyldipentylsilane and diethyl(di-2-methylpropyl)silane are non-limiting examples of silahydrocarbons that are synthesized via hydrosilylation with the iron catalysts disclosed in U.S. Pat. No. 9,371,399.

(CH₃)₂SiH₂ +2 C₅H₁₀→(CH₃)₂Si(C₅H₁₁)₂ (Dimethyldipentylsilane)

(C₂H₅)₂SiH₂+2 (CH₃)₂CH═CH₂ →[(CH₃)₂CH₂CH₂]₂Si(C₂H₅)₂ (Diethyl(di-2-methyl-propyl)silane)

Grignard reactions may also be utilized to prepare silahydrocarbons. Grignard reactions involve the reaction of an alkyl silyl halide, e.g., a silylchloride, with an alkyl magnesium chloride. The alkyl groups may be provided as desired to produce the desired silahydrocarbon.

Alkyllithium reactions involve the reaction of an alkyl lithium compound with an alkyl silyl hydride or an alkyl silyl chloride.

Additional synthetic methods for preparing silahydrocarbons include, but are not limited to, the following: Silahydrocarbons synthesized by organofluorophosphonium salt catalyzed Hydrosilylation of trialkyl hydrosilane with olefins. (M. Perez, L. J. Hounjet, C. B. Caputo, R. Dobrovetsky, D. W. Stephan, J. Am. Chem. Soc., 2013, 135, 18308-18310); Silahydrocarbons synthesized by triethylborane catalyzed Hydrosilylation with various alkenes. (M. J. Palframan, A. F. Parsons, P. Johnson, Synlett, 2011, 2811-2814); Silahydrocarbons that are synthesized by a zinc-catalyzed nucleophilic substitution reaction of chlorosilanes with organomagnesium reagents. (K. Murakami, H. Yorimitsu, K. Oshima, J. Org. Chem., 2009, 74, 1415-1417); Silahydrocarbons that are synthesized by palladium-Catalyzed Cross-Coupling of Silyl Electrophiles with Alkylzinc Halides ((Silyl-Negishi Reaction) (A. P. Cinderella, B. Vulovic, D. A. Watson, J. Am. Chem. Soc., 2017, 139, 7741-7744); Silahydrocarbons that are synthesized via reaction of trimethylsilylsodium or potassium with alkyl halides (H. Sakurai & F. Kondo, J. Organometallic Chem., 1975, 92, C46-C48); and Silahydrocarbons that are synthesized by nickel catalyst (NiBr₂ diglyme) couplings of alkyl bromides with nucleophilic silicon reagents. (C. K. Chu, Y. Liang, G. C. Fu, J. Am. Chem. Soc., 2016, 138, 6404-6407).

The solvent can comprise the silahydrocarbon in a suitable concentration as desired for, or may be suitable for, an intended use or application. In one embodiment, the solvent is a solution that is 100% silahydrocabon (or a combination of two or more silahydrocarbons). In one embodiment the solvent comprises the silahydrocarbon in an amount of from about 40% to about 100%, from about 50% to about 90%, or from about 60% to about 80% by weight based on the total weight of the solvent. It will be appreciated that the concentration of the silahydrocarbon in the solvent can be selected on the particular type of personal care formulation in which the solvent is employed.

When the solvent comprises less than 100% silahydrocarbon, the silahydrocrbon may be mixed in a suitable diluent or carrier. Examples of suitable diluents or carriers include, but are not limited to, paraffinic hydrocarbons, (e.g., C5-C40 hydrocarbons, C8-C20 isoparaffins), C1-C40 alcohols, oils (e.g., naturally occurring or synthetic glyceryl esters or fatty acids or triglycerides), alkyl ethers, and/or monoesters of carboxylic acids. Some examples of suitable carriers include, but are not limited to, Isododecane, Isoparaffin, Petroleum Jelly, Castor Oil, Soybean Oil, Jojoba Oil, Avocado Oil, Isopropyl Palmitate, PDMS-10 cst Fluid, cyclosiloxane, MQ Resin, Cetyl Alcohol, Ethanol, Isopropyl Alcohol, Propylene Glycol, Octyl Methoxycinnamate, Octyl Salicylate, Tocopheryl Acetate, Retinyl palmitate.

The amount of silahydrocarbon in the personal care composition can be selected as desired for a particular purpose or intended application. In one embodiment, the silahydrocarbon content of the personal care composition is from about 1% to about 80% by weight, from about 5% to about 60% by weight, from about 10% to about 50% by weight, from about 15% to about 40% by weight, or from about 20% to about 30% by weight based on the total weight of the personal care composition.

The personal care composition comprises at least one personal care component. The personal care component can be a functional agent or material suitable to provide a desired type of personal care product.

Examples of suitable personal care components, in embodiments, include, but are not limited to, a gel forming polymer, a film forming polymer, an emulsion, an elastomer, a resin, a wax, an oil, an alcohol, an ester, a pigment, a bio-active agent, or a combination of two or more thereof.

The film-forming agents can be selected as desired depending on the type of personal care composition and intended use. Examples of suitable film-forming agents include, but are not limited to, organosiloxane resins, hydrocarbon polymers, hydrocarbon polymer containing heteroatoms, fluorine atom containing hydrocarbon polymers, and clays.

Useful organosiloxane film-forming agents may comprise combinations of R^{a}₃SiO_{1/2}, R^{a}₂SiO, R^{a}SiO_{3/2}, and SiO₂units in ratios to each other that satisfy the relationship R^{a}ₙSiO_{(4-n)/}2 where n is a value between 0 and 1.50 and R^{a} is independently selected from methyl, trifluoromethyl, phenylenemethyl and phenyl groups.

Useful hydrocarbon polymer film-forming agents in accordance with the present invention may be selected from, but are not limited to, polybutene, polyisobutene, polycyclopentadiene, isododecane, isohexadecane, petroleum jelly, mineral oil, or a combination of two or more thereof.

The hydrocarbon polymers containing at least one heteroatom that are useful as film-forming agents in accordance with the present invention may be selected from, but are not limited, to polyvinylpyrrolidone (PVP) copolymer, polyvinylpyrrolidone (PVP)/eicosene Copolymer, PVP/hexadecene Copolymer, dimehticone copolymer, acrylate copolymer, and polyvinyl alcohol copolymer. The polymer can also be a crosslinked polymer such as, but not limited to, crosslinked vinyl dimethicone polymer.

Useful film-forming clays in accordance with the present invention may include, but are not limited to, various modified and unmodified clays including, but not limited to, cloisite 30B, momtmorillite (NaMMT), LDH and bentonite.

Still other examples of useful film-forming agents include natural waxes, polymers such as polyethylene polymers, copolymers of PVP, ethylene vinyl acetate, dimethicone gum, resins such as shellac, polyterpenes, and the like.

Waxes suitable for use herein include, but are not limited to, animal, natural, and/or synthetic waxes. Examples of suitable waxes include, but are not limited to, beeswax, paraffin, rice bran wax, candelilla wax, carnauba wax, ozokerite wax, derivatives thereof. It will be appreciated that a single type of wax or a combination of two or more waxes can be employed in the personal care composition.

Resins suitable for the use in the personal care composition can be selected as desired for the particular type of personal care composition and intended use. Examples of suitable resins include, but are not limited to, silicone resin, vinyl resin, urea resin, melamine resin, phenol resin, epoxy resin, or a combination of two or more thereof.

The personal care component can be an emulsion. The emulsion can be a lamellar emulsion, a microemulsion, or a nanoemulsion. In addition, the emulsion may be a fluid simple emulsion, a fluid multiple emulsion, a rigid simple emulsion, or a rigid multiple emulsion. The simple emulsion or multiple emulsion may comprise a continuous aqueous phase containing dispersed lipid vesicles or oil droplets, or a continuous fatty phase dispersed lipid vesicles or water droplets. The emulsion can be in the form of oil-in-water, water-in-oil, and anhydrous emulsions, where in each of these emulsions the silicone phase may be either the discontinuous phase or the continuous phase, as well as multiple emulsions, such as, for example, oil-in-water-in-oil emulsions and water-in-oil-in-water emulsions.

The personal care component can be selected from a gel forming polymer. The gel forming polymer can be selected as desired for a particular purpose or intended application. Examples of suitable gel forming polymers include, but are not limited to, a crosslinked silicone gel, a crosslinked acrylamide gel, a crosslinked hydrocarbon gel, a functionalized cellulose gel, or a combination of two or more thereof.

The personal care component can be selected from an oil. The oil may be chosen based on the type of personal care composition or intended end use. The oil can be selected from a natural or synthetic oil. Oils can include, but are not limited to, a vegetable oil or a mineral oil. Examples of suitable oils include, but are not limited to, jojoba oil, Macadamia nut oil, almond oil, avocado oil, sunflower oil, thistle oil, castor oil, apricot kernel oil, peach kernel oil, coconut oil, palm oil, olive oil, sesame oil, soybean oil, and one or more active substance oils, preferably selected from the group argon oil, Rosehip Oil (Rose hip seed oil), Evening primrose oil, Borage seed oil, Camellias oil, Amaranth seed oil, Babussaöl, Rice bran oil, Grapeseed oil, walnut oil, wheat germ oil, cottonseed oil, calendula oil, hemp oil, currant seed oil, kukui nut oil, laurel oil, poppy seed oil and black cumin oil.

The personal care component can be selected from an alcohol. The alcohol is not particularly limited and can be selected as desired based on the type of personal care composition and the intended use. Examples of suitable alcohols include, but are not limited to, glycerin, glycols, butylene glycol, propylene glycol, pentylene glycol, cetyl alcohol, propylene glycol ethanol, polyvinyl alcohol, phenoxyethanol, isononyl alcohol, neopentyl alcohol, or combinations of two or more thereof.

Pigments suitable for use herein are all inorganic and organic colors/pigments. These are usually aluminum, barium or calcium salts or lakes. A lake is a pigment that is extended or reduced with a solid diluent or an organic pigment that is prepared by the precipitation of a water-soluble dye on an adsorptive surface, which usually is aluminum hydrate. A lake also forms from precipitation of an insoluble salt from an acid or basic dye. Calcium and barium lakes are also used herein. Other colors and pigments can also be included in the compositions, such as pearls, titanium oxides, Red 6, Red 21, Blue 1, Orange 5, and Green 5 dyes, chalk, talc, iron oxides, boron nitride, zinc oxides, magnesium oxides, and titanated micas.

The personal care composition of the present invention may optionally contain vitamins or skin nourishing agents, hair nourishing agents, antidandruff additives, antibacterial, and antifungal agents. Some of the preferred agents are ceramides, hyaluronic acid, panthenol, peptides (copper hexapeptide-3), AHAs (lactic acid), retinols (retinyl palmitate)-vitamin A derivatives, vitamin C (1-ascorbic acid), BHAs (salicylic acid), niacinamide, tocopherol, teas (green tea, white tea, red tea), soy and other plant derivatives, isoflavones (grape seed extract), argireline, acai berry, pyridinethione salts, 1-hydroxy-2-pyrrolidone derivatives, 2,2′-dithiobis(pyridine-N-oxide), trihalocarbamides, triclosan, azole compounds such as climbazole, ketoconazole, clotrimazole, econazole, isoconazole and miconazole, selenium sulphides, extracts of one or more non-photosynthetic, non-fruiting filamentous bacteria, zinc pyrithione, piroctone olamine, selenium disulphide, sulphur and coal tar.

The personal care composition of the present invention is preferentially incorporated into a carrier, specifically, a volatile carrier which quickly volatilizes after application. The volatile carriers of the present invention are selected from the group consisting of volatile hydrocarbons, volatile silicones and mixtures thereof. Hydrocarbon oils useful in the present invention include those having boiling points in the range of 60-260° C., more preferably hydrocarbon oils having from about C₈ to about C₂₀ chain lengths, most preferably C8 to C20 isoparaffins. Most preferred are selected from the group consisting of isododecane, isohexadecane, isoeocosane, 2,2,4-trimethylpentane, 2,3-dimethylhexane and mixtures thereof. Preferred volatile silicone fluids include cyclomethicones having 3, 4 and 5 membered ring structures corresponding to the formula (R^{b}₂SiO)ₓ, where x is from about 3 to about 6 or linear siloxanes corresponding to the formula (R^{b}₃SiO(R^{b}₂SiO)SiR^{b}₃ where R^{b}is independently selected from a C1-C10 alkyl.

A thickening polymer may be useful in the present invention. The expression “thickening polymer” refers to a polymer having, in solution or in dispersion containing 1% by weight of active material in water or in ethanol at 250° C., a viscosity greater than 0.2 poise at a shear rate of 10 rad/second. The viscosity can be measured with a HAAKE RS600 viscometer from THERMO ELECTRON. This viscometer is a controlled-stress viscometer with cone-plate geometry (for example, having a diameter of 60 mm and an angle of 1°). Examples of thickeners include, but are not limited to, associative thickeners; crosslinked acrylic acid homopolymers; crosslinked copolymers of (meth)acrylic acid and of (C₁-C₆)alkyl acrylate; nonionic homopolymers and copolymers containing ethylenically unsaturated monomers of ester and/or amide type; ammonium acrylate homopolymers or copolymers of ammonium acrylate and of acrylamide; (meth)acrylamido(C₁-C₄)alkylsulphonic acid homopolymers and copolymers; crosslinked methacryloyl(C₁-C₄)alkyltri(C₁-C₄)alkylammonium homopolymers and copolymers. Particulate thickeners may also be used. Also, naturally derived polymers and polymers produced by fermentations may be used such as polysaccharide gums, xanthan gum, pullulan gum, sclerotium gum, carrageenan gum, locust bean gum, alginate, gellan gum, cellulose, carboxymethylcellulose, hydroxyethylcellulose, pectins, starch, chitosan, gelatin and their combination.

Particulates may also be used in combination with the personal care composition of the present invention. Particulates may be organic or inorganic particles. Examples of inorganic particles include, but are not limited to, microparticles composed of titanium oxide, titanated mica, zirconium oxide, zinc oxide, cerium oxide, magnesium oxide, barium sulfate, calcium sulfate, magnesium sulfate, calcium carbonate, magnesium carbonate, talc, cleaved talc, mica, kaolin, sericite, muscovite, synthetic mica, phlogopite, lepidolite, biotite, lithia mica, silicic acid, silicon dioxide, fumed silica, hydrous silicon dioxide, aluminum silicate, magnesium silicate, aluminum magnesium silicate, calcium silicate, barium silicate, strontium silicate, metal tungstenate salts, hydroxyapatite, vermiculite, higilite, bentonite, montmorillonite, hectorite, zeolite, ceramics, dicalcium phosphate, alumina, aluminum hydroxide, boron nitride, boron nitride or glass, Example of organic particles include powders composed of a polyamide, polyacrylic acid/acrylic acid ester, polyester, polyethylene, polypropylene, polystyrene, styrene/acrylic acid copolymer, divinylbenzene/styrene copolymer, polyurethane, vinyl resin, urea resin, melamine resin, benzoguanamine, polymethylbenzoguanamine, tetrafluoroethylene, polymethylmethacrylate (such as poly(methyl methacrylate)), cellulose, silk, nylon, phenol resin, epoxy resin or polycarbonate.

Useful additives include pH adjusters/buffering agents and chelating agents such as, but not limited to, ammonium hydroxide, sodium hydroxide, potassium hydroxide, C₁₂-C₁₅ alkyl benzoate, citric acid, glycolic acid, lactic acid, sodium citrate, triethanolamine, trolamine, disodium EDTA, edetate disodium, pentasodium pentetate, tetrasodium EDTA, trisodium EDTA.

Fragrance ingredients may be incorporated in the personal care composition of the present invention, e.g., diacetyl, isoamyl acetate, benzaldehyde, cinnamic aldehyde, ethyl propionate, methyl anthranilate, limonene, ethyl decadienoate, allyl hexanoate, ethyl maltol, ethyl vanillin, methyl salicylate, clary extract, eucalyptus oil, grapefruit oil, labdanum oil, masking fragrance, matricaria oil, nopyl acetate, phenoxyethanol, rosewood oil, ylang oil, and perfume oils. There may also be mentioned as perfume oils mixtures of natural and/or synthetic aromatic substances. Natural aromatic substances include, e.g., extracts from blossom (lilies, lavender, roses, jasmine, neroli, ylang-ylang), from stems and leaves (geranium, patchouli, petitgrain), from fruit (aniseed, coriander, carraway, juniper), from fruit peel (bergamot, lemons, oranges), from roots (mace, angelica, celery, cardamom, costus, iris, calmus), from wood (pinewood, sandalwood, guaiacum wood, cedarwood, rosewood), from herbs and grasses (tarragon, lemon grass, sage, thyme), from needles and twigs (spruce, pine, Scots pine, mountain pine), from resins and balsams (galbanum, elemi, benzoin, myrrh, olibanum, opoponax). Animal raw materials also come into consideration, for example civet and castoreum. Typical synthetic aromatic substances include, e.g., products of the ester, ether, aldehyde, ketone, alcohol or hydrocarbon type. Aromatic substance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate.

Ethers include, e.g., benzyl ethyl ether; the aldehydes include, e.g., linear alkanals having from 8 to 18 hydrocarbon atoms, citral, citronellal, citronellyl oxyacetaldehyde, cyclamen aldehyde, hydroxycitronellal, lilial and bourgeonal; ketones include, e.g., ionones, isomethylionone and methyl cedryl ketone; alcohols include, e.g., anethol, citronellol, eugenol, isoeugenol, geraniol, linalool, phenyl ethyl alcohol and terpinol; and, hydrocarbons include, e.g., terpenes and balsams.

Mixtures of these and other aromatic substances may be used in selected combinations as desired to produce an especially attractive scent. Ethereal oils of relatively low volatility, which are chiefly used as aroma components, are also suitable as perfume oils, e.g., sage oil, chamomile oil, clove oil, melissa oil, oil of cinnamon leaves, lime blossom oil, juniper berry oil, vetiver oil, olibanum oil, galbanum oil, labolanum oil and lavandin oil. Preference is given to the use of bergamot oil, dihydromyrcenol, lilial, lyral, citronellol, phenyl ethyl alcohol, α,α-hexyl cinnamaldehyde, geraniol, benzyl acetone, cyclamen aldehyde, linalool, boisambrene forte, ambroxan, indole, hedione, sandelice, lemon oil, tangerine oil, orange oil, allyl amyl glycolate, cyclovertal, lavandin oil, muscatel sage oil, a-damascone, bourbon geranium oil, cyclohexyl salicylate, vertofix coeur, iso-E-Super, Fixolide NP, evernyl, iraldein gamma, phenylacetic acid, geranyl acetate, benzyl acetate, rose oxide, romillat, irotyl and floramat alone or in admixture with one another.

Flavor ingredients may be used in the personal care composition of the present invention such as, but not limited to, stearyl glycyrrhetinate, menthol, cinnamyl alcohol, acetic acid, ascorbic acid, citric acid, fumaric acid, lactic acid malic acid, phosphoric acid, tartaric acid, fruit, and plant extracts.

Skin protectants and humectants may be used in the personal care composition of the present invention such as, but not limited to, dimethicone, petrolatum, glycerin, ammonium lactate, lanolin, methyl gluceth-20, PEG-20, sorbitol, 1,2,6 hexanetriol, butylene glycol, dipropylene glycol, glycerin, hexylene glycol, panthenol, phytantriol, panthenol, propylene glycol, sodium PCA, sorbitol, triethylene glycol, polyglyceryl sorbitol, glucose, fructose, polydextrose, potassium pyrrolidone carboxylic acid (pca), urea, hydrogenated honey, hyaluronic acid, inositol, hexanediol beeswax, hexanediol beeswax, hydrolyzed elastin, hydrolyzed collagen, hydrolyzed silk, hydrolyzed keratin, erythritol, capryl glycol, and the like.

Hair conditioning agents may be used herein, e.g., hydrocarbons, silicone fluids and cationic materials. Suitable hydrocarbons can be either straight or branched chain and can contain from about 10 to about 16, and preferably from about 12 to about 16, carbon atoms. Examples of suitable hydrocarbons include, e.g., decane, dodecane, tetradecane, tridecane, and mixtures thereof. Examples of suitable silicone fluid conditioning agents include, e.g., linear and cyclic polydimethylsiloxanes, phenyl and alkyl phenyl silicones and silicone copolyols. Cationic conditioning materials agents useful herein include, e.g., quaternary ammonium salts and the salts of fatty amines.

Nail conditioning agents may be incorporated in the present invention, e.g., adipic acid, fumaric acid, tricyclodecane dimethanol copolymer, AMP-isostearoyl hydrolyzed silk, angelica furcijuga flower/leaf/stem extract, r-Bacillus Licheniformis keratinase, bifida/panax ginseng root cell culture extract ferment filtrate, bis-aminopropyl dimethicone/IPDI copolymer, bis-dicaprolactone ethoxyacrylate IPDI, bis-hEMA IPDI, boswellia carterii gum extract, boswellia serrata gum extract, calcium hydrolyzed collagen, capryloyl methionine/silk amino acids methyl esters, capryloyl serine/silk amino acid methyl esters, caulerpa eacemosa extract, citrus aurantium amara (bitter orange) fruit juice extract, commelina communis flower/leaf/stem extract, commiphora myrrha resin extract, dechloro dihydroxy difluoro ethylcloprostenolamide, deoxyglutamyl fructose, dicaprolactone ethoxyacrylate hema ipdi, dicapryl succinate, dimethyl urea, dipentaerythrityl hexaacrylate, dipentaerythrityl pentaacrylate, echinacea angustifolia root extract,fragaria ananassa (strawberry) seed oil, fumaric acid/phthalic anhydride/tricyclodecanedimethanol copolymer, Ginkgo Biloba nut extract, glycidoxypropyl trimethoxysilane, honey powder, hydnocarpus pentandrus kernel oil, hydrogenated acetophenone/oxymethylene copolymer, hydrolyzed collagen, hydrolyzed keratin, hydrolyzed vinylacetate/vinyl acetoacetate copolymer, isatis tinctoria root extract, kigelia africana bark extract, leucanthemum vulgare seed extract, leuconostoc/aloe barbadensis leaf/sorbus aucuparia fruit ferment filtrate, lobelia inflata extract, lupinus texensis seed extract, lycium barbarum fruit extract, mentha aquatica extract, methacryloylethyl phosphate, methylene glycol, 2-methylpropanal, Momordica Charantia extract, narcissus pseudo-narcissus (daffodil) root extract, opuntia tuna leaf extract, opuntia vulgaris fruit extract, palmitoyl oligopeptide-70, palmitoyl serine/silk amino acids methyl esters, Phaseolus Vulgaris (kidney bean) extract, phlox drummondii seed extract, polyacrylate-12, polyacrylate-30, polyester-18, rosa (american beauty) extract, rosa borboniana extract, rosa roxburghii seed oil, rudbeckia hirta seed extract, silver carp extract, sodium calcium zinc phosphate, sorbus aucuparia seed oil, r-spider polypeptide-1, r-spider polypeptide-2, undecenoyl serine/silk amino acid methyl esters, vaccinium myrtillus leaf extract, vinyl alcohol/vinylformamide copolymer, viscum album (mistletoe) extract, and the like.

Cationic polymers can be used in the personal care composition of the present invention, e.g., cationic guar gum derivatives such as guar hydroxypropyltrimonium chloride and hydroxypropyl guar hydroxypropyltrimonium chloride, available as the Jaguar@ series from Rhone-Poulenc.

Ultraviolet light absorbers (UV absorbers) may be useful utilized in the personal care composition herein to protect the composition from chemical or physical deterioration induced by ultraviolet light. UV absorbers, like sunscreen agents, have the ability to convert incident ultraviolet radiation into less damaging infrared radiation (heat). Suitable UV absorbers include e.g., acetaminosalol, allantoin PABA, benzalphthalide, benzophenones such a benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, and phenone-12, benzotriazolyl dodecyl p-cresol, 3-benzylidene camphor, benzylidenecamphor hydrolyzed collagen sulfonamide, benzylidene camphor sulfonic acid, benzyl salicylate, bis-ethylhexyloxyphenol methoxyphenyl triazine, bornelone, bumetrizole, butyl methoxydibenzoylmethane, butyl PABA, Callophyllum inophyllum Seed Oil, Camellia Sinensis leaf extract, carotenoids, ceria/silica, ceria/silica talc, cinoxate, dea-methoxycinnamate, dibenzoxazoyl naphthalene, di-t-butyl hydroxybenzylidene camphor, diethylhexyl butamido triazone, diethylhexyl 2,6-naphthalate, digalloyl trioleate, diisopropyl methyl cinnamate, 1-(3,4-dimethoxyphenyl)-4,4-dimethyl-1,3-pentanediene, dimethyl PABA ethyl cetearyldimonium tosylate, dimorpholinopyridazinone, diphenyl carbomethoxy acetoxy naphthopyran, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, esculin, ethyl dihydroxypropyl PABA, ethyl diisopropylcinnamate, ethylhexyl dimethoxybenzylidene dioxoimidazolidine propionate, ethylhexyl dimethyl PABA, ethylhexyl ferulate, ethylhexyl methoxycinnamate, ethylhexyl salicylate, ethylhexyl triazone, ethyl methoxycinnamate, ethyl PABA, ethyl urocanate, etocrylene, ferulic acid, 4-(2-beta-glucopyranosiloxy)propoxy-2-hydroxybenzophenone, glyceryl ethylhexanoate dimethoxycinnamate, glyceryl PABA, glycol salicylate, hexanediol salicylate, homosalate, hydrolyzed lupine protein, isoamyl p-methoxycinnamate, isopentyl trimethoxycinnamate trisiloxane, isopropylbenzyl salicylate, isopropyl dibenzoylmethane, isopropyl methoxycinnamate, menthyl anthranilate, menthyl salicylate, 4-methylbenzylidene camphor, methylene bis-benzotriazolyl tetramethylbutylphenol, octocrylene, octrizole, PABA, PEG-25 PABA, pentyl dimethyl PABA, phenylbenzimidazole sulfonic acid, pinus pinaster bark extract, polyacrylamidomethyl benzylidene camphor, polysilicone-15, potassium methoxycinnamate, potassium phenylbenzimidazole sulfonate, Red petrolatum, sodium benzotriazolyl butylphenol sulfonate, sodium isoferulate, sodium phenylbenzimidazole sulfonate, sodium urocanate, Spirulina platensis powder, tea-phenylbenzimidazole sulfonate, TEA-salicylate, terephthalylidene dicamphor sulfonic acid, tetrabutyl phenyl hydroxybenzoate, titanium dioxide, tocotrienols, triPABA panthenol, urocanic acid, vinyl acetate/crotonates/methacryloxybenzophenone-1 copolymer and Vitis vinifera (grape) seed extract, and polymeric beads or hollow spheres as SPF enhancers. The combination of the UV-absorbers such as those described above with SPF enhancers such as styrene/acrylate copolymers silica beads, spheroidal magnesium silicate, spherical polyamide powder such as n-lactam polymer (Orgasol® range, elf atochem) cross-linked polymethylmethacrylates (pmma; micopearl m305 seppic), may enhance the UV protection. Holosphere additives (Sunspheres® ISP, Silica Shells Kobo.) deflect radiation and the effective path length of the photon is therefore increased. (EP0893119). Some beads, provide a soft feel during spreading. Moreover, the optical activity of such beads, e.g., Micropearl M305, can modulate skin shine by eliminating reflection phenomena and indirectly may scatter UV light.

The personal care composition of the present invention may also contain one or more known and conventional plasticizers in order to improve the flexibility and cosmetic properties of the resulting formulation. Plasticizers are frequently used to avoid brittleness and cracking of film formers and include, e.g., lecithin, polysorbates, dimethicone copolyol, glycols, citrate esters, glycerin, and dimethicone. One skilled in the art may routinely vary the amount of plasticizer(s) depending on the formulation of a particular personal care composition and the properties desired.

The personal care composition of the present invention is preferentially formulated with a carrier, specifically, a volatile carrier which quickly volatilizes after application of the composition. Useful conventional volatile carriers may be selected from the group consisting of volatile hydrocarbons, volatile silicones and mixtures thereof. The Silahydrocarbons of this invention can also function as volatile carriers.

The personal care composition of the present invention may optionally include up to 50 parts by weight of blocking and/or absorbing sunscreen agents. Blocking sunscreen agents are generally inorganic, such as various cerium oxides, chromium oxides, cobalt oxides, iron oxides, red petrolatum, silicone- and other treated titanium dioxides, titanium dioxide, zinc oxide, and/or zirconium oxide, BaTiO₃, CaTiO₃, SrTiO₃ and SiC. Absorbing sunscreen agents, which are usually organic species, include, but are not limited to, UV-A absorbers, which generally absorb radiation in the 320 to 400 nm region of the ultraviolet spectrum, e.g., anthranilates, benzophenones and dibenzoyl methanes; and, UV-B absorbers, which generally absorb radiation in the 280 to 320 nm region of the ultraviolet spectrum, e.g., p-aminobenzoic acid derivatives, camphor derivatives, cinnamates and salicylates.

Specific examples of organic sunscreen agents include p-aminobenzoic acid, avobenzone cinoxate, dioxybenzone, homosalate, menthyl anthranilate, octocrylene, octyl methoxycinnamate, octyl salicylate, oxybenzone, padimate, phenylbenzimidazole sulfonic acids, sulisobenzone, trolamine salicylate, aminobenzoic acid, amyldimethyl p-aminobenzoic acid, diethanolamine p-methoxycinnamate, digalloyl trioleate, 2-ethylhexyl-2-cyano-3,3-diphenylacrylate, ethylhexyl p-methoxycinnamate, 2-ethylhexyl salicylate, glyceryl aminobenzoate, homomethyl salicylate, homosalate, 3-imidazol-4-ylacrylic acid and the ethyl ester thereof, methyl anthranilate, octyldimethyl PABA, 2-phenylbenzimidazole-5-sulfonic acid and salts, sulisobenzone, triethanolamine salicylate, N,N,N-trimethyl-4-(2-oxoborn-3-ylidene methyl)anillinium methyl sulfate, aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammonium)-benzyliden-boman-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′ dimethoxybenzophenone, 2-hydroxy-4-n-octoxybenzophenone, 2-hydroxy-4-methoxy-4′-methoxybenzophenone, ca-(2-oxobom-3-ylidene)-tolyl-4-sulfonic acid and soluble salts thereof, 3-(4′-sulfo)benzyliden-boman-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris-(2′-ethylhexyl-1′-oxycarbonyl)-anilinol 1,3,5-triazine, 2-(p-(tert-butylamido)anilinol-4,6-bis-(p-(2′-ethylhexyl 1′-oxycarbonyl)anilinol 1,3,5-triazine, 2,4-bis{1,4-(2-ethylhexyloxy)-2-hydroxyl-phenyl}-6-(4-methoxyphenyl)-1,3,5-triazine, the polymer of N-(2 et 4)-(2-oxoborn-3-yliden)methylbenzyl acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (drometrizole trisiloxane), solubilized 2,2′-methylene-bis-1,6-(2H-benzotriazol-2-yl)-4-(1,1,3,3-tetramethylbutyl)phenol, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, and combinations comprising at least one of the foregoing sunscreen agents.

The personal care component can be selected as desired based on the type of personal care product for which the composition is to be employed (i.e., for the particular intended end use). Personal care compositions in accordance with the present invention include, but are not limited to deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-nail-and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, drug delivery systems for topical application of medicinal compositions that are to be applied to the skin and combinations comprising at least one of the foregoing personal care applications.

The personal care compositions can be prepared by mixing the selected personal care component(s) with the solvent comprising the silahydrocarbon. Mixing can be carried out by any suitable means and under suitable conditions (e.g., temperature, time, etc.) as needed to effectively blend the materials to form the composition.

The following examples are intended to illustrate aspects and embodiments of the present technology. All parts and percentages are by weight and all temperatures are in Celsius (° C.) unless explicitly stated otherwise. All patents, other publications, and U.S. patent applications referred to in the instant application are incorporated herein by reference in their entireties.

### Examples

All air- and moisture-sensitive manipulations were carried out using a standard vacuum line and a round bottom flask under an inert atmosphere of purified nitrogen. A grade of Karstedt's Catalyst containing 2 wt. % platinum in Xylene was purchased from Sigma Aldrich and used as received for conducting the hydrosilylation experiments of Examples 1-7. Chloroform-d was purchased from Cambridge Isotope Laboratories and used as received for all NMR characterization purpose.

### Synthetic Example 1: Trimethyloctylsilane

1-octene (14.56 g, 130.0 mmol) and Karstedt's Catalyst (0.0073 g) were taken in a 250 ml round-bottom flask fitted with reflux condenser, magnetic stirrer, thermocouple and nitrogen inerting. The flask was placed in an oil bath maintained at 30° C. Then, trimethylsilane gas was passed through the reaction mixture in a controlled manner to maintain an average 20° C. exotherm for about 3 hours. The completion of the reaction was indicated by disappearance of terminal olefin bond peak between (5-6 ppm) in the proton NMR spectrum. Then the pure product was collected as colorless liquid through vacuum (10 mmHg, 100° C.) distillation. Yield=18.2 g (82.7%), ¹H NMR (400 MHz, CDCl₃): δ 0.0 ppm (9H, s), 0.5 (2H, t), 0.9 (3H, t), 1.3 (12H, m).

### Synthetic Example 2: Trimethyldodecylsilane

1-dodecene (21.84 g, 130 mmol) and Karstedt's Catalyst (0.011 g) were taken in a 250 ml round-bottom flask fitted with a reflux condenser, magnetic stirrer, thermocouple and nitrogen inertion. The mixture was placed into an oil bath maintained at 30° C. Then, trimethylsilane gas was passed through the reaction mixture in a controlled manner to maintain an average 20° C. exotherm for about 3 hours. The completion of the reaction was indicated by disappearance of terminal olefin bond peak (5-6 ppm) in the proton NMR studies. The pure product was collected as colorless liquid through vacuum distillation (10 mmHg, 140° C.). Yield=23.0 g (78.8%)¹H NMR (400 MHz, CDCl₃): δ 0.0 ppm (9H, s), 0.5 (2H, t), 0.9 (3H, t), 1.3 (20H, m).

### Synthetic Example 3: Bis(trimethylsilyl) octane

1,7-Octadiene (8.46 g, 76.9 mmol) and Karstedt's Catalyst (0.004 g) were taken in a 250 ml round-bottom flask fitted with reflux condenser, magnetic stirrer, thermocouple and nitrogen inertion. The mixture was placed into an oil bath maintained at 30° C. Then, trimethylsilane gas was passed through the reaction mixture in a controlled manner to maintain an average 20° C. exotherm for about 3 hours. The completion of the reaction was indicated by disappearance of terminal olefin bond peak (5-6 ppm) in the proton NMR studies. The pure product was collected as colorless liquid through vacuum distillation. Yield=15.2 g (65.3%),¹H NMR (400 MHz, CDCl₃): δ 0.0 ppm (18H, s), 0.5 (4H, t), 1.3 (12H, m).

### Synthetic Example 4: 1-(Dimethyl-n-butylsilyl) octane

1-Octene (14.56 g, 130.0 mmol) and Karstedt's Catalyst (0.0073 g) were taken in a 250 ml round-bottom flask fitted with reflux condenser, magnetic stirrer, thermocouple and nitrogen inerting. The mixture was placed into an oil bath maintained at 40° C. Added dropwise dimethylchlorosilane (9.4 g, 100 mmol) and continue to stir the reaction for 12 hours. The completion of the reaction was indicated by disappearance of terminal olefin bond peak (5-6 ppm) in the proton NMR studies. The unreacted dimethylchlorosilane and isomerized olefin by product were removed by vacuum (10 mmHg, 120° C.) stripping of the reaction mixture. Butyl lithium (6.4 g, 100 mmol) was added to the reaction mixture and continued to stir for 6 hours. The clear liquid isolated from lithium chloride by product using filtration technique. The vacuum distillation ((10 mmHg, 120° C.)) of the supernatant liquid afforded pure product as colorless liquid. Yield=11.2 g (39%).

### Synthetic Example 5: Triethylhexylsilane

1-hexene (5.46 g, 65 mmol), triethylsilane (5.8 g, 50 mmol) and Karstedt's Catalyst (0.0055 g) were taken in a 250 ml round-bottom flask fitted with reflux condenser, magnetic stirrer, thermocouple, and nitrogen inerting. The mixture was placed into an oil bath maintained at 80° C. and continued to stir for 16 hours. The completion of the reaction was indicated by disappearance of terminal olefin bond (5-6 ppm) in the proton NMR studies. Then the pure product was collected as colorless liquid through vacuum distillation (10 mmHg, 120° C.). Yield: 6.3 g, (56.0%), ¹H NMR (400 MHz, CDCl₃): δ 0.5 (8H, m), 0.9 (12H, m) 1.3 (8H, m).

### Synthetic Example 6: Triethyl (3-methyl-1-butyl) silane

3-Methyl-1-butene (4.55 g, 65 mmol), triethylsilane (5.8 g, 50 mmol) and Karstedt's Catalyst (0.0051 g) were taken in a 250 ml round-bottom flask fitted with reflux condenser, magnetic stirrer, thermocouple and nitrogen inerting. The mixture was placed into an oil bath maintained at 60° C. and continued to stir for 16 hours. The completion of the reaction was indicated by disappearance of terminal olefin bond (5-6 ppm) in the proton NMR studies. Then the pure product was collected as colorless liquid through vacuum distillation (5 mmHg, 120° C.). Yield: 5.7 g. (55%).

### Synthetic Example 7: Triethyloctyl silane

1-Octene (7.28 g, 65.0 mmol), triethylsilane (5.8 g, 50.0 mmol) and Karstedt's Catalyst (0.0065 g) were taken in a 250 ml round-bottom flask fitted flask fitted with reflux condenser, magnetic stirrer, thermocouple and nitrogen inerting. The mixture was placed into an oil bath maintained at 60° C. and continued to stir for 16 hours. The completion of the reaction was indicated by disappearance of terminal olefin bond (5-6 ppm) in the proton NMR studies. Then the pure product was collected as colorless liquid through vacuum distillation. Yield=7.4 g, (56.5%), ¹H NMR (400 MHz, CDCl₃): δ 0.4 (8H, m), 0.8 (12H, m) 1.3 (12H, m).

### Synthetic Example 8: 2,4,4-Trimethylpentyltriethylsilane

Triethylsilane (10 g, 0.09 mole) and 2,4,4-trimethyl-1-pentene (59 g, 0.53 mole) were introduced into a 250 ml, N₂-flushed, 3-neck round bottom flask fitted with a heating mantle, magnetic stirrer, reflux condenser, addition funnel and thermocouple. The reflux condenser and thermocouple were affixed via a Claisen connection, thereby enabling the third neck of the flask to be sealed with a serum cap. The stirred contents were heated to 80° C. Karstedt's catalyst (25 μL, 2.25 wt. % complex in xylene) and 1,5-cyclooctadiene (10 μL) were then added. The remaining triethylsilane (49 g, 0.42 mole) was added dropwise from the addition funnel during the following 30 minutes as heating was continued. Refluxing was observed at 110° C. and the temperature was maintained at that point overnight. The reaction mixture was brown with no visible solid formation. It was stripped in vacuo to remove the unreacted reagents. The residue was analyzed by GC-MS, which indicated the presence of (CH₃)₃CCH₂CH(CH₃)CH₂Si(C₂H₅)₃ (Mass 228). Major mass spectrometry fragments were m/e 199 (loss of C₂H₅), 115 (loss of C₈H₁₇), 101, 87 and 57.

### Synthetic Example 9: Dibutyldimethylsilane

The reaction was carried out in a Radley's reactor having a mechanical stirrer, fitted with a condenser, addition funnel and temperature probe. Before reaction, the reactor was degassed three times to remove any moisture with alternate vacuum and N₂ cycles. Later, the reactor was kept under dry N₂. The reactor was charged with BuLi (315.3g; 0.9 equiv. of 2M solution in cyclohexane) by cannular transfer. Then it was heated to 60° C. with stirring. Dichlorodimethyl silane (50 g; 1 equiv) along with anhydrous THF was introduced to the reaction mixture through an addition funnel under nitrogen atmosphere. The reaction was started by the slow addition of dichlorodimethylsilane to control the exotherm. The progress of the reaction was accompanied by formation LiCl salt. After complete addition of silane, the stirred reaction mixture was maintained at 60° C. for additional 1hour. After complete consumption of dichlorodimethylsilane, the reaction was quenched by the addition of saturated NaHCO₃(40 wt. %) solution after cooling the reaction vessel to ice cold condition. The product layer separated from the aqueous layer with hexane was washed thrice with water and once with 40% brine solution. The resulting solution was then dried over anhydrous sodium sulfate and vacuum stripped under reduced pressure (25° C., 0-1 mbar). The final product was recovered from the reaction mixture by fractional distillation (packed column with SS Dixon packings) under reduced pressure (45° C.; 0-1 mbar). A pure fraction of dibutyldimethylsilane was collected at 45° C. at vacuum of 0-1 mbar. The product obtained was analyzed by ¹H-NMR {CDCl₃, 0ppm; 6H(Si—CH₃); 0.5 ppm 4H(Si—CH₂); 0.9 ppm; 6H(Si—CH₂—CH₂—CH₂—CH₃), 1.3 ppm; 8H(Si—CH₂—CH₂—CH₂)}; ²⁹Si-NMR (δ 2.5 ppm) and GC/MS (99.998% purity; m/z 172.2).

### Synthetic Example 10: Tributylmethylsilane

The reaction was carried out in a Radley's reactor having a mechanical stirrer, fitted with a condenser, addition funnel, and temperature probe. Before reaction, the reactor was degassed three times to remove any moisture with alternate vacuum and N₂ cycles. Later the reactor was kept under dry N₂ atmosphere. The reactor was charged with BuLi (375.9 g: 2.9 equiv. of 2M solution in cyclohexane) by cannular transfer. It was then heated to 60° C. under stirring condition. The trichloromethyl silane (50 g; 1 equiv) along with anhydrous THF was introduced to the reaction mixture thorough an addition funnel under nitrogen atmosphere. The reaction was initiated by the slow addition of trichloromethylsilane while controlling the exotherm. The progress of the reaction was indicated by gradual formation LiCl salt. After complete addition of silane, the reaction was continued to stir at 60° C. for 1 hour. The completion of the reaction was monitored by checking the acidic pH of the reaction mixture. After complete consumption of trichloromethylsilane, the reaction was quenched by the addition of saturated NaHCO₃(40 wt. %) solution after cooling the reaction vessel to ice cold condition. The product layer separated from the aqueous layer with hexane was washed thrice with water and once with 40% brine solution. Later the collected materials were dried over anhydrous sodium sulfate and the solvent was vacuum stripped under reduced pressure (25° C., 0-1 mbar). The final product was then extracted from the crude mixture by fractional distillation (packed column with SS Dixon packings) under reduced pressure (75° C.; 0-1 mBar). Pure fraction of triibutylmethylsilane was collected at temperature 75° C. at vacuum of 0-1 mBar. The product obtained is analyzed by ¹H {CDCl₃, 0ppm; 3H(Si—CH₃); 0.5 ppm 6H(Si—CH₂); 0.9 ppm; 9H(Si—CH₂—CH₂—CH₂—CH₃), 1.3 ppm; 12H(Si—CH₂—CH₂—CH₂)};), ²⁹Si-NMR (δ 2.6 ppm) and GC/MS (99.990% purity; m/z 214.2).

### Formulations Examples

Personal care formulations were prepared as described below, and the properties of the material tested as indicated. Properties that were evaluated include coefficient of friction, volatility, and spreading/spreadability. These properties are indicative of sensory properties associated with the compositions.

Coefficient of Friction (COF): Lubricity profile provides very useful insight into the post-delivery sensorial properties of the material used in personal care application (H&PC Today -Household and Personal Care Today, Vol. 9 nr. 3 May/Jun. 2014). Lubricity, which is closely associated with a material's smoothness and slipperiness properties, has often been used as a tool to quantify the overall sensory of the personal care materials where higher lubricity often correlates to better sensory attribute to the consumers. The lubricity profile of silahydrocarbons was evaluated by measuring their coefficients of friction using a substrate friction accessory attached to the MTT175 Diastron Instrument (Reference: https://www.diastron.com/app/uploads/2018/06/Dia-Stron-MTT175-Brochure-V2.pdf, accessed on Apr. 6, 2020). The pre-hydrated in-vitro skins were used to simulate the real life human skin conditions. Experimentally, 50 μL of each silahydrocarbon compound was uniformly applied onto the 2 cm×4 cm in-vitro skin placed on the horizontal plate of the substrate friction accessory. The plate was then subjected to movement at a speed of 20 mm/minute against the probe under a vertical weight of 200 g. The data were then processed using UV2000win software provided with MTT175 by Dia-Stron Ltd. to obtain the coefficient of friction value (vide infra Table 16)

Volatility: The volatility of silahydrocarbon materials of the present invention was evaluated by isothermal thermogravimetric analysis as described in H&PC Today -Household and Personal Care Today, Vol. 9 nr. 3 May/Jun. 2014. Approximately 15 mg of each material was warmed to 30° C. and evaporated for 120 min under a lateral nitrogen flow of 25 mL/min, after which the percent loss was calculated. The results were plotted and presented as a chart.

Spreading Test: In personal care, the selection of a volatile carrier can have a significant impact on a product's ease of application. Higher spreadability correlates with increased ease of use of the end product. The spreading properties of silahydrocarbon materials were evaluated against various benchmark materials according a modified procedure described in the literature (H&PC Today-Household and Personal Care Today, Vol. 9 nr. 3 May/Jun. 2014). Thus, 10 μL of each compound was dropped onto the hydrated in-vitro skin, at 20-25° C. and 75-80% percent relative humidity. After 5 minutes of diffusion, the perimeter of the spreading area was marked to enable the spreading area measurements. The results are reported based on triplicate measurements and summarized in Table 15 herein below.

Example 11: Anhydrous sunscreen spray containing silahydrocarbons (CF1 and CF2 are comparative examples, F1 represents the formulation examples employing silahydrocarbons).

TABLE 1

| Ingredient | | | | | |
|---|---|---|---|---|---|
| Trade Name | INCI/IUPAC Name | Supplier | CF1 | CF2 | F1 |
| SF1318 | Diisostearoyl | Momentive | 8.00 | 8.00 | 8.00 |
| | Trimethylolpropane | | | | |
| | Siloxy Silicate | | | | |
| Crodamol AB | C12-15 Alkyl Benzoate | Croda | 10.00 | 10.00 | 10.00 |
| SF 1550 | Phenyl Trimethicone | Momentive | 15.00 | 15.00 | 15.00 |
| SS 4230 | Cyclopentasiloxane (and) | Momentive | 5.00 | 5.00 | 5.00 |
| | Trimethylsiloxysilicate | | | | |
| Uvinul MC 80 | Octyl Methoxycinnamate | BASF | 7.00 | 7.00 | 7.00 |
| UVINUL N 539 T | Octocrylene | BASF | 8.00 | 8.00 | 8.00 |
| SilSoft ETS | 1,1,1,2,3,3,3- | Momentive | 47.00 | 0.00 | 0.00 |
| | Heptamethyl 2- | | | | |
| | ethyltrisiloxane | | | | |
| SF 1202 | Decamethylpentasiloxane | Momentive | 0.00 | 47.00 | 0.00 |
| Synthetic | Dibutyldimethylsilane | Momentive | 0.00 | 0.00 | 47.00 |
| Example No. 9 | | | | | |
| | Total | | 100 | 100 | 100 |

Procedure and Results

All ingredients were weighed and mixed at 500-800 rpm at ambient temperature until homogeneous (approximately 30 minutes). It was observed that the formulation containing dibutyldimethylsilane (Synthetic Example 9) had a post smooth skin feel comparable to that of Cyclopentasiloxane.

Example 12: Aqua gel crème formulation containing silahydrocarbons (CF3 is a comparative example and F2 is the formulation employing a silahydrocarbon).

TABLE 2

| | Ingredient | INCI/ | | | |
|---|---|---|---|---|---|
| Phase | Trade Name | Chemical Name | Supplier | CF3 | F2 |
| A | Water | Aqua | | 84.20 | 84.20 |
| | Glycerin | Glycerin | Sigma Aldrich | 3.00 | 3.00 |
| | Disodium | Disodium EDTA | Sigma Aldrich | 0.05 | 0.05 |
| | EDTA | | | | |
| | Silsoft 876 | PEG-12 Dimethicone | Momentive | 0.30 | 0.30 |
| | fluid | | | | |
| | Softouch CC | Boron Nitride | Momentive | 0.50 | 0.50 |
| | 6097 | | | | |
| | Sepigel 305 | Polyacrylamide (and) | Seppic | 1.00 | 1.00 |
| | | C13-14 Isoparaffin | | | |
| | | (and) Laureth-7 | | | |
| B | SF 1202 | Cyclopentasiloxane | Momentive | 10.00 | 0.00 |
| | Synthetic | Dibutyldimethylsilane | Momentive | 0.00 | 10.00 |
| | Example | | | | |
| | No. 9 | | | | |
| | Euxy1 9010 | Phenoxyethanol (and) | Schülke & | 0.70 | 0.70 |
| | | Ethylhexylglycerin | Mayr | | |
| | Fragrance | Fragrance | Goldfield | 0.25 | 0.25 |
| | | | Fragrances | | |

| | | |
|---|---|---|
| Total | 100 | 100 |

Procedure and Results.

All ingredients in Phase A were weighed and mixed at 800-1000 rpm until homogeneous. Phase B ingredients were added individually one after the other to the homogenized Phase A. Mixing continued until ahomogeneous gel crème structure formed.

It was observed that the formulation with dibutyldimethylsilane had better spreadability, absorption, post-moisturization and skin smoothness over D5. In addition, dibutyldimethylsilane was found to give body/richness to the formulation.

Example 13: Foundation formulation containing silahydrocarbons (CF4 is a comparative formulation and F3 is a formulation example of the present invention.)

TABLE 3

| | Ingredient Trade | INCI Name/ | | | |
|---|---|---|---|---|---|
| Phase | Name | Chemical Name | Supplier | CF4 | F3 |
| A | Silsoft 1202 | Cyclopentasiloxane | Momentive | 21.00 | 0.00 |
| | Synthetic Example | Dibutyldimethylsilane | Momentive | 0.00 | 21.00 |
| | 10 | | | | |
| | Silsoft 034 fluid | Caprylyl Methicone | Momentive | 5.00 | 5.00 |
| | Titanium dioxide | Titanium Dioxide (C.I. | Koel Colors | 8.00 | 8.00 |
| | | 77891) | | | |
| | Yellow iron oxide | Iron Oxides (C.I. | Koel Colors | 1.00 | 1.00 |
| | | 77492) | | | |
| | Red iron oxide | Iron Oxides (C.I. | Koel Colors | 0.36 | 0.36 |
| | | 77491) | | | |
| | Iron Oxide black | Iron Oxides (C.I. | Koel Colors | 0.14 | 0.14 |
| | | 77499) | | | |
| B | Silsoft 1540 fluid | Cyclopentasiloxane | Momentive | 3.50 | 3.50 |
| | | (and) PEG/PPG-20/15 | | | |
| | | Dimethicone | | | |
| | Bentone^{†} Gel VS5- | Cyclopentasiloxane | Elementis | 3.00 | 3.00 |
| | PCV | (and) | | | |
| | | Disteardimonium | | | |
| | | Hectorite (and) | | | |
| | | Propylene Carbonate | | | |
| C | Water | Water | | 52.50 | 52.50 |
| | Velvesil E-Gel | Dimethicone/Vinyl | Momentive | 3.00 | 3.00 |
| | PMF emulsion | Dimethicone | | | |
| | | Crosspolymer (and) | | | |
| | | Dimethicone (and) | | | |
| | | Isohexadecane (and) | | | |
| | | Cetearyl Methicone | | | |
| | Tween 20 | Polysorbate-20 | Croda | 1.50 | 1.50 |
| | Sodium Chloride | Sodium Chloride | Sigma Aldrich | 0.50 | 0.50 |
| | DMDM Hydantoin | DMDM Hydantoin | Salicylates & | 0.50 | 0.50 |
| | | | Chemicals | | |

| | | |
|---|---|---|
| Total | 100 | 100 |

Procedure and Results:

Ingredients in Phase A were weighed and mixed thoroughly using homogenizer at 1500-2000 rpm until homogeneous and complete color development. The components in Phase B were mixed at 1500-2000 rpm and mixed with Phase A until homogeneous. The components in Phase C were mixed at 1500-2000 rpm until homogeneous. Phase C was added slowly to the combined Phase A and B while homogenizing at 1500-2000 rpm. Addition was slow to ensure that the water was fully incorporated into Phase A and B. After complete addition of Phase C, homogenization was continued at 3500-4000 rpm for 2 minutes. It was observed that Foundation formulation with dibutyldimethylsilane was comparable to that with D5 with respect to quick spreading, absorption, post skin feel and pigment blend.

Compatibility Study:

Procedure and Results:

The subject materials were added in 50:50 ratio to a vial and mixed using a vortex shaker for 5 minutes, then observed visually for any phase separation. Observations reported in Table 4 show that the silahydrocarbon prepared in synthetic Example 9 showed equivalent compatibility to D5, whereas the silahydrocaron prepared in synthetic Example 1 has better compatibility and transparency than D5.

TABLE 4
Compatibility performance of silahydrocarbons
with personal care ingredients

| | Synthetic | Synthetic | |
|---|---|---|---|
| | Example 1 | Example 9 | |
| | (Trimethyloctyl- | Dibutyldimethyl | Control |
| PC Ingredients | silane) | Silane | (D5) |
| Isododecane | Yes | Yes | Yes |
| Petroleum Jelly | Yes | NA | Yes |
| Castor Oil | Yes | No | No |
| Soybean Oil | Yes | NA | Yes |
| Jojoba Oil | Yes | Yes | Yes |
| Avocado Oil | Yes | NA | Yes |
| Isopropyl Palmitate | Yes | Yes | Yes |
| Isopropyl Palmitate | Yes | NA | Yes |
| PDMS-10 cst Fluid | Yes | Yes | Yes |
| SF1202 | Yes | Yes | Yes |
| MQ Resin | Yes | NA | Yes |
| Cetyl Alcohol | Yes | NA | Yes |
| Ethanol | Yes | Yes | Yes |
| Isopropyl Alcohol | Yes | NA | Yes |
| Propylene Glycol | Partial | NA | Partial |
| Ozokerite | Yes | NA | Yes |
| Candelilla (Euphorbia | Yes | NA | Yes |
| Cerifera) Wax | | | |
| Beeswax | Yes | NA | Yes |
| Carnauba (Copernicia | Yes | NA | Yes |
| Cerifera) Wax | | | |
| Octyl Methoxycinnamate | Yes | NA | Yes |
| Octyl Salicylate | Yes | NA | Yes |
| Tocopheryl Acetate | Yes | NA | Yes |
| Retinyl palmitate | Yes | NA | Yes |

NA = Not analyzed

TABLE 5
Spreading performance of silahydrocarbons

| | Area | Standard |
|---|---|---|
| Compound Name | (mm²) | Deviation |
| Synthetic Example 1 [Trimethyloctylsilane] | 1385 | 0 |
| Synthetic Example 3 [Bis(trimethylsilyl) octane] | 1134 | 0.78 |
| Synthetic Example 5 [Triethylhexylsilane] | 1278 | 0.26 |
| Synthetic Example 7 [Triethyloctyl silane] | 1364 | 0.26 |
| Control 2 [Cyclopentasiloxane, D5] | 1114 | 0.26 |

As shown in Table 5, the silahydrocarbons trimethyloctylsilane, bis(trimethylsilyl)octane, triethylhexylsilane and triethyloctylsilane exhibit better spreadability compared to the benchmark cyclopentasiloxane (D5).

TABLE 6
Coefficient of Friction (COF) for silahydrocarbons molecules

| Compound Name | COF | STDV |
|---|---|---|
| Synthetic Example 1 [Trimethyloctylsilane] | 0.44 | 0.03 |
| Synthetic Example 2 [Trimethyldodecylsilane] | 0.58 | 0.08 |
| Synthetic Example 3 [Bis(trimethylsilyl) octane] | 0.74 | 0.03 |
| Synthetic Example 5 [Triethylhexylsilane] | 0.73 | 0.06 |
| Synthetic Example 6 [Triethyl (3-methyl-1-butyl) silane] | 0.77 | 0.02 |
| Synthetic Example 7 [Triethyloctyl silane] | 0.43 | 0.05 |
| Control [Cyclopentasiloxane, D5] | 1.12 | 0.11 |

As shown in Table 6, the silahydrocarbons trimethyloctylsilane, trimethyldodecylsilane, bis(trimethylsilyl) octane, triethylhexylsilane, triethyl (3-methyl-1-butyl) silane and triethyloctyl silane exhibit lower COF compared to the benchmark cyclopentasiloxane (D5). Therefore, these silahydrocarbons are expected to deliver better sensory compared to cyclopentasiloxane.

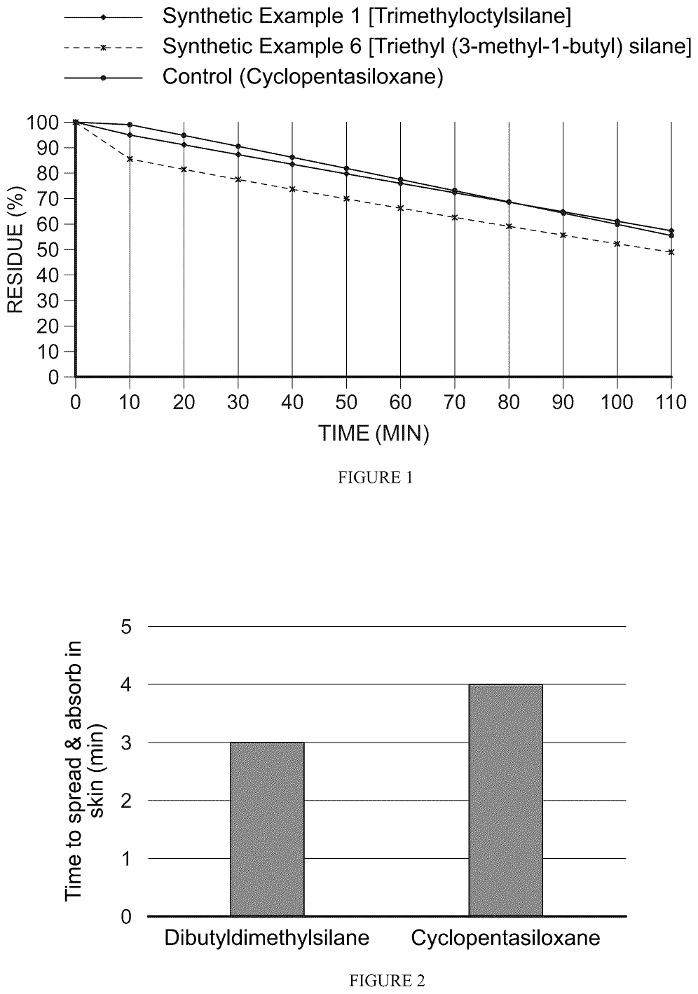

Volatility data of silahydrocarbons: FIG. 1 shows that trimethyloctylsilane and triethyl (3-methyl-1-butyl) silane exhibit volatility profile equivalent to cyclopentasiloxane.

Spreadability and Absorption properties on Skin

The diameter of the circle formed by the material was measured and it was observed that dibutyldimethylsilane (Synthetic example 9) spreads in a larger diameter than D5. The larger diameter indicates not only a lower surface tension, but also that formulations containing this raw material would spread more easily upon skin, rub-in would require less effort and offer a smooth application.

FIG. 2 shows the time required for the raw materials to completely dry off. Dibutyldimethylsilane took less time to dry off than that required for Cyclopentasiloxane. Reduced drying time implies that when used in a personal care formulation, dibutyldimethylsilane can increase the capacity of that formulation to reduce the overall sensation of tackiness and wetness.

The present invention provides a solution to the long-standing need for replacing the cyclosiloxanes in the compositions used for personal care applications. The silahydrocarbons of the present invention have superior compatibility, spreading performances, coefficient of friction, and smoothness without compromising on volatility characteristic of cyclosiloxanes.

The foregoing description identifies various, non-limiting embodiments of silahydrocarbons when used in personal care compositions. Modifications may occur to those skilled in the art and to those who may make and use the invention. The disclosed embodiments are merely for illustrative purposes and not intended to limit the scope of the invention or the subject matter set forth in the claims.

1. A personal care composition comprising (i) a solvent comprising at least one silahydrocarbon, and (ii) at least one personal care component.

2. The personal care composition of claim 1, wherein the silahydrocarbon is selected from a compound of the formula (I), formula (II), or a mixture of such silahydrocarbons:
R¹R²R³R⁴Si (I); or
R⁵R⁶R⁷Si(Q)SiR⁸R⁹R¹⁰ (II)
wherein R¹, R², R³, R⁴, R⁵, R⁶, R⁷, R⁸, R⁹, and R¹⁰ are each independently selected from an aliphatic hydrocarbyl group having 1 to 30 carbon atoms, an aryl group having 6 to 30 carbon atoms, an alkaryl having 7 to 30 carbon atoms, and a cycloaliphatic hydrocarbyl group having from 4 to 30 carbon atoms, and Q is a group bridging between silicon atoms.

3. The personal care composition of claim 2, wherein Q is selected from a straight-chained or branched alkylene group having from 0 to 14 carbon atoms, a cyclic hydrocarbon having 4 to 14 carbon atoms, and an aryl group having 6 to 14 carbon atoms.

4. The personal care composition of claim 1, wherein the silahydrocarbon has a total number of carbons of about 30 or less.

5. The personal care composition of claim 1, wherein the silahydrocarbon has a total number of carbons of about 20 or less.

6. The personal care composition of claim 1, wherein silahydrocarbon has a total number of carbon atoms of from about 5 to about 30.

7. The personal care composition of claim 1, wherein the silahydrocarbon has a total number of carbon atoms of from about 7 to about 20.

8. The personal care composition of claim 1, wherein the silahydrocarbon is selected from Trimethyl(butyl)silane; Trimethyl(pentyl)silane;
Trimethyl(hexyl)silane; Trimethyl(heptyl)silane; Trimethyl(octyl)silane; Trimethyl(nonyl) silane; Trimethyl(decyl)silane; Trimethyl(undecyl)silane; Trimethyl(dodecyl)silane; Ethyldimethyl(butyl)silane; Ethyldimethyl(pentyl)silane; Ethyldimethyl (hexyl)silane; Ethyldimethyl (heptyl)silane; Ethyldimethyl (octyl)silane; Ethyldimethyl (nonyl) silane; Ethyldimethyl (decyl)silane; Ethyldimethyl (undecyl)silane; Ethyldimethyl (dodecyl)silane; Diethylmethyl(butyl)silane; Diethylmethyl (pentyl)silane; Diethylmethyl (hexyl)silane; Diethylmethyl (heptyl)silane; Diethylmethyl (octyl)silane; Diethylmethyl (nonyl) silane; Diethylmethyl (decyl)silane; Diethylmethyl (undecyl)silane; Diethylmethyl (dodecyl)silane; Triethyl (butyl); Triethyl (pentyl)silane; Triethyl (hexyl)silane; Triethyl (heptyl)silane; Triethyl (octyl)silane; Triethyl (nonyl) silane; Triethyl (decyl)silane; Triethyl (undecyl)silane; Triethyl (dodecyl)silane; Tetraethylsilane; Tetrabutylsilane [R¹=R²=R³=R⁴=butyl](16 carbon atoms); Tributylmethylsilane; Tributylethylsilane; Dibutyldimethylsilane; Dibutyldiethylsilane; Tetrapropylsilane; Tripropylmethylsilane; Tripropylethylsilane; Dipropyldimethylsilane; Dipropyldiethylsilane; 2,4,4-Trimethylpentyltriethylsilane; Hexamethyldisilane, Bis(trimethylsilyl)methane, Bis(trimethylsilyl)ethane; Bis(trimethylsilyl)butane; Bis(trimethylsilyl)pentane; Bis(trimethylsilyl)hexane; Bis(trimethylsilyl)heptane; Bis(trimethylsilyl) octane; Bis(triethylsilyl)butane; Bis(triethylsilyl)pentane; Bis(triethylsilyl)hexane; Bis(triethylsilyl)heptane; Bis(triethylsilyl)octane; 1-Dimethylbutylsilyl 8-trimethylsilyl octane; or a combination of two or more thereof.

9. The personal care composition of 1, wherein the silahydrocarbon is present in the formulation in an amount of from about 40% to about 100% by weight based on the total weight of the composition.

10. The personal care composition of claim 1, wherein the silahydrocarbon is present in an amount of from about 1% to about 80% by weight based on the total weight of the personal care composition.

11. The personal care composition of claim 1, wherein the silahydrocarbon is present in an amount of from about 5% to about 60% by weight based on the total weight of the personal care composition.

12. The personal care composition of 1, wherein the silahydrocarbon is present in an amount of from about 10% to about 50% by weight based on the total weight of the personal care composition.

13. The personal care composition of 1, wherein at least one personal care component is selected from a gel forming polymer, a film forming polymer, an emulsion, an elastomer, a resin, a wax, an oil, an alcohol, an ester, a pigment, a bio-active agent, a sunscreen agent, or a combination of two or more thereof.

14. The personal care composition of claim 13, wherein the gel forming polymer is selected from a crosslinked silicone gel, a crosslinked acrylamide gel, a crosslinked hydrocarbon gel, a functionalized cellulose gel, or a combination of two or more thereof.

15. The personal care composition of claim 13, wherein the wax is selected from beeswax, paraffin, rice bran wax, candelilla wax, carnauba wax, ozokerite wax, or a combination of two or more thereof.

16. The personal care composition of 13, wherein the emulsion is selected from a lamellar emulsion, a microemulsion, a nanoemulsion, a fluid simple emulsion, a fluid multiple emulsion, a rigid simple emulsion, or a rigid multiple emulsion.

17. The personal care composition of claim 16, wherein the emulsion is an oil-in-water emulsion or a water-in-oil emulsion.

18. The personal care composition of claim 13, wherein the hydrocarbon is selected from polybutene, polyisobutene, polycyclopentadiene, isododecane, isohexadecane, petroleum jelly, mineral oil, or a combination of two or more thereof.

19. The personal care composition of claim 13, wherein the oil is selected from jojoba oil, Macadamia nut oil, almond oil, avocado oil, sunflower oil, thistle oil, castor oil, apricot kernel oil, peach kernel oil, coconut oil, palm oil, olive oil, sesame oil, soybean oil, and one or more active substance oils, preferably selected from the group argon oil, Rosehip Oil (Rose hip seed oil), Evening primrose oil, Borage seed oil, Camellias oil, Amaranth seed oil, Babussaöl, Rice bran oil, Grapeseed oil, walnut oil, wheat germ oil, cottonseed oil, calendula oil, hemp oil, currant seed oil, kukui nut oil, laurel oil, poppy seed oil, black cumin oil, or a combination of two or more thereof.

20. The personal care composition of claim 13, wherein the alcohol is selected from glycerin, glycol, butylene glycol, propylene glycol, pentylene glycol, cetyl alcohol, propyelene glycol ethanol, polyvinyl alcohol, phenoxyethanol, isononyl alcohol, neopentyl alcohol, or a combination of two or more thereof

21. The personal care composition of claim 13, wherein the personal care component is selected from a dimethicone polymer, a dimethicone copolymer, a polyvinyl alcohol copolymer, a polyacrylate copolymer, a polyvinyl pyrrolidone copolymer, a crosslinked vinyl dimethicone polymer, a silicone elastomer, a polymethylmethacrylate elastomer, ispropyl myristate, isopropyl palmitate, or a combination of two or more thereof.

22. The personal care composition of claim 13, wherein the personal care component is selected from a vitamin, skin nourishing agent, hair nourishing agent, antidandruff additive, antibacterial, antifungal agent, or combination of two or more thereof.

23. The personal care composition of claim 22, wherein the a vitamin, skin nourishing agent, hair nourishing agent, antidandruff additive, antibacterial, or antifungal agent is selected form ceramides, hyaluronic acid, panthenol, peptides (copper hexapeptide-3), AHAs (lactic acid), retinols (retinyl palmitate)-vitamin A derivatives, vitamin C (1-ascorbic acid), BHAs (salicylic acid), niacinamide, tocopherol, teas (green tea, white tea, red tea), soy and other plant derivatives, isoflavones (grape seed extract), argireline, acai berry, pyridinethione salts, 1-hydroxy-2-pyrrolidone derivatives, 2,2′-dithiobis(pyridine-N-oxide), trihalocarbamides, triclosan, an azole compound, selenium sulphides, extracts of one or more non-photosynthetic, non-fruiting filamentous bacteria, zinc pyrithione, piroctone olamine, selenium disulphide, sulphur, coal tar, or a combination of two or more thereof.

24. The personal care composition of claim 13, wherein the sunscreen agent is selected from p-aminobenzoic acid, avobenzone cinoxate, dioxybenzone, homosalate, menthyl anthranilate, octocrylene, octyl methoxycinnamate, octyl salicylate, oxybenzone, padimate, phenylbenzimidazole sulfonic acids, sulisobenzone, trolamine salicylate, aminobenzoic acid, amyldimethyl p-aminobenzoic acid, diethanolamine p-methoxycinnamate, digalloyl trioleate, 2-ethylhexyl-2-cyano-3,3-diphenylacrylate, ethylhexyl p-methoxycinnamate, 2-ethylhexyl salicylate, glyceryl aminobenzoate, homomethyl salicylate, homosalate, 3-imidazol-4-ylacrylic acid and the ethyl ester thereof, methyl anthranilate, octyldimethyl PABA, 2-phenylbenzimidazole-5-sulfonic acid and salts, sulisobenzone, triethanolamine salicylate, N,N,N-trimethyl-4-(2-oxoborn-3-ylidene methyl)anillinium methyl sulfate, aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammonium)-benzyliden-boman-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′ dimethoxybenzophenone, 2-hydroxy-4-n-octoxybenzophenone, 2-hydroxy-4-methoxy-4′-methoxybenzophenone, ca-(2-oxoborn-3-ylidene)-tolyl-4-sulfonic acid and soluble salts thereof, 3-(4′-sulfo)benzyliden-boman-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris-(2′-ethylhexyl-1′-oxycarbonyl)-anilinol 1,3,5 -triazine, 2-(p-(tert-butylamido)anilinol-4,6-bis-(p-(2′-ethylhexyl 1′-oxycarbonyl)anilinol 1,3,5-triazine, 2,4-bis{1,4-(2-ethylhexyloxy)-2-hydroxyl-phenyl}-6-(4-methoxyphenyl)-1,3,5-triazine, the polymer of N-(2 et 4)-(2-oxoborn-3-yliden)methylbenzyl acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (drometrizole trisiloxane), solubilized 2,2′-methylene-bis-1,6-(2H-benzotriazol-2-yl)-4-(1,1,3,3-tetramethylbutyl)phenol, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, or a combination of two or more thereof.

25. The personal care composition of claim 13, wherein the pigment is selected from a pearl, titanium oxide, Red 6, Red 21, Blue 1, Orange 5, Green 5, chalk, talc, iron oxide, boron nitride, zinc oxide, magnesium oxide, titanated mica, or a combination of two or more thereof.

26. The personal care composition of claim 1, wherein the personal care composition is selected from deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail- and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, drug delivery systems for topical application of medicinal compositions that are to be applied to the skin.

27. A method of treating a biological substrate comprising applying the personal care composition of claim 1 to a surface of the biological substrate.

28. The method of claim 27, wherein the biological substrate is skin or hair.
